(19) United States
(12) Patent Application Publication
(10) Pub. No.: US 2026/0107971 A1
(43) Pub. Date: Apr. 23, 2026

(54) AEROSOL GENERATING MATERIAL IN DISCRETE PORTIONS COMPRISING A SUBSTITUTED 3-(1-METHYLPYRROLIDIN-2-YL)PYRIDINE COMPOUND

(71) Applicant: RAI STRATEGIC HOLDINGS, INC., Winston-Salem, NC (US)

(72) Inventors: Kyle Ford, Pfafftown, NC (US); Brandon S. Darrow, Winston-Salem, NC (US); Michael R. Galloway, Winston-Salem, NC (US); Gary M. Dull, Lewisville, NC (US)

(21) Appl. No.: 19/361,573

(22) Filed: Oct. 17, 2025

Related U.S. Application Data
(60) Provisional application No. 63/708,870, filed on Oct. 18, 2024.
(60) Provisional application No. 63/880,497, filed on Sep. 12, 2025.

(51) Int. Cl. A24B 15/167; A24B 15/30; A24B 15/32; A24B 15/40; A24B 15/42; A24F 40/20; A61K 31/4427; A61K 31/465

(52) U.S. Cl. CPC A24B 15/167; A24B 15/301; A24B 15/32; A24B 15/403; A24B 15/406; A24B 15/42; A24F 40/20; A61K 31/4427; A61K 31/465

(57) ABSTRACT

Disclosed herein is a consumable including one or more discrete portions of aerosol generating material. The aerosol generating material includes an aerosol former, a binder, and an active agent, wherein the active agent includes at least a substituted 3-(1-methylpyrrolidin-2-yl)pyridine, 3-(azetidin-2-yl)pyridine, or 3-(azetidin-2-ylmethoxy)pyridine. Further provided is a non-combustible aerosol provision system including the consumable.

### CROSS-REFERENCE TO RELATED APPLICATIONS

This application claims priority to U.S. Provisional Application No. 63/708,870, filed Oct. 18, 2024, and claims priority to U.S. Provisional Application No. 63/880,497 filed Sep. 12, 2025, each of which is incorporated herein by reference in their entirety and for all purposes.

### FIELD OF THE DISCLOSURE

The present disclosure relates to aerosol generating materials and articles for use in an aerosol provision system, to aerosol provision systems including such materials and articles, and to methods for forming the aerosol generating materials and articles.

### BACKGROUND

Smoking articles such as cigarettes, cigars and the like burn tobacco during use to create tobacco smoke. Alternatives to these types of combustible articles release an inhalable aerosol or vapor by releasing compounds from a substrate material by heating without burning. These may be referred to as non-combustible smoking articles or aerosol generating articles.

One example of such a product is a heating device which release compounds by heating, but not burning, a solid aerosolizable material. This solid aerosolizable material may, in some embodiments, contain a tobacco material. The heating volatilizes at least one component of the material, typically forming an inhalable aerosol. These products may be referred to as heat-not-burn devices, tobacco heating devices or tobacco heating products. Various different arrangements for volatilizing at least one component of the solid aerosolizable material are known.

### BRIEF SUMMARY

The present disclosure is generally directed to a consumable for use in a non-combustible aerosol provision system. The consumable comprises one or more discrete portions of aerosol generating material, the aerosol generating material comprising an aerosol former, a binder, and an active agent, the active agent comprising at least a substituted 3-(1-methylpyrrolidin-2-yl)pyridine, 3-(azetidin-2-yl)pyridine, or 3-(azetidin-2-ylmethoxy)pyridine. The consumable may be incorporated into a non-combustible aerosol provision system.

The present disclosure includes, without limitation, the following embodiments.

Embodiment 1: A consumable comprising one or more discrete portions of aerosol generating material, the aerosol generating material comprising an aerosol former, a binder, and an active agent, the active agent comprising at least a compound having a structure according to Formula I, Formula II, or Formula III:

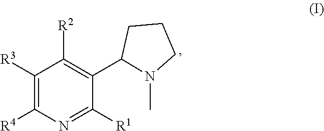

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or

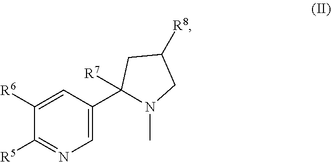

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C1-C3 alkyl; and
   at least one of R⁷ and R⁸ is not hydrogen; or

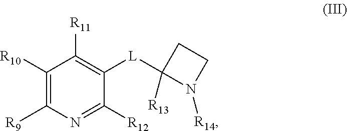

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

Embodiment 2: The consumable of embodiment 1, wherein the active agent comprises a compound having a structure according to Formula I, wherein R¹, R², and R³ are each H.

Embodiment 3: The consumable of embodiment 1 or 2, wherein the active agent comprises a compound having a structure according to Formula I, wherein R⁴ is optionally substituted C₁-C₆ alkyl, F, Cl, Br, OCH₃, OEt, or CN.

Embodiment 4: The consumable of embodiment 3, wherein R⁴ is C₁-C₃ alkyl, such as wherein R⁴ is CH₃.

Embodiment 5: The consumable of embodiment 1, wherein the active agent comprises a compound having a structure according to Formula II, wherein R⁵ and R⁶ are H; R⁷ is CH₃; and R⁸ is H.

Embodiment 6: The consumable of embodiment 1, wherein the active agent comprises a compound having a structure according to Formula II, wherein R⁵, R⁶ and R⁷ are H, and R⁸ is CH₃.

Embodiment 7: The consumable of any one of embodiments 1-6, wherein the active agent comprises a compound having a structure according to Formula I or Formula II, wherein each discrete portion comprises from about 0.01 to about 5 mg of the active agent.

Embodiment 8: The consumable of any one of embodiments 1-6, wherein the active agent comprises a compound having a structure according to Formula I or Formula II, and wherein the aerosol generating material comprises from about 0.1 to about 5 wt % of the active agent.

Embodiment 9: The consumable of any one of embodiments 1-8, wherein the discrete portions of aerosol generating material are located on a carrier.

Embodiment 10: The consumable of embodiment 9, wherein the carrier is formed from metal foil, paper, carbon paper, grease-proof paper, ceramic, a carbon allotrope, plastic, cardboard, wood, or a combinations thereof.

Embodiment 11: The consumable of any one of embodiments 1-10, comprising at least two discrete portions of aerosol generating material, and wherein a total amount of the active agent in any given discrete portion is within 10% of a total amount of active agent in any of the other discrete portions.

Embodiment 12: The consumable of any one of embodiments 1-11, wherein the aerosol generating material comprises from about 1 to about 80% by weight of the aerosol former.

Embodiment 13: The consumable of any one of embodiments 1-12, wherein the aerosol former comprises glycerol, propylene glycol, 1,3-propanediol, diethylene glycol, triethylene glycol, tetraethylene glycol, 1,3-butylene glycol, erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, or a combination thereof.

Embodiment 14: The consumable of any one of embodiments 1-13, wherein the aerosol generating material comprises from about 0.5% to about 60% of the binder by weight.

Embodiment 15: The consumable of any one of embodiments 1-14, wherein the binder comprises an alginate, a pectin, a starch, a cellulose derivative, a gum, silica, clay, polyvinyl alcohol, or a combination thereof.

Embodiment 16: The consumable of any one of embodiments 1-15, wherein the aerosol generating material further comprises a filler in an amount by weight up to about 20%.

Embodiment 17: The consumable of embodiment 16, wherein the filler is a fibrous organic material, a cellulosic material, or a combination thereof.

Embodiment 18: A non-combustible aerosol provision system comprising a non-combustible aerosol provision device and the consumable of any one of embodiments 1 to 17.

Embodiment 19: The non-combustible aerosol provision system of embodiment 18, wherein the non-combustible aerosol provision device is a heat-not-burn device.

Embodiment 22: The consumable of embodiment 1, wherein the active agent has a structure according to Formula II; optionally, wherein R⁵ is optionally substituted C₁-C₆ alkyl, F, Cl, Br, OCH₃, OCH₂CH₃, or CN; and R⁶ is H; or optionally, wherein R⁵ is H; and R⁶ is optionally substituted C₁-C₆ alkyl, F, Cl, Br, OCH₃, OCH₂CH₃, or CN.

Embodiment 23: The consumable of embodiment 1, wherein the active agent has a structure according to Formula III; optionally, wherein R⁹ is H or CH₃; and R¹⁰, R¹¹, and R¹² are each H.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example embodiments, should be viewed as combinable, unless the context of the disclosure clearly dictates otherwise.

### BRIEF DESCRIPTION OF THE DRAWINGS

Having thus described aspects of the disclosure in the foregoing general terms, reference will now be made to the accompanying figures, which are not necessarily drawn to scale, and wherein:

FIG. 1 is a cross-sectional schematic representation of an aerosol provision system according to a non-limiting embodiment of the disclosure, comprising an aerosol provision device and an aerosol generating article, the device comprising a plurality of heating elements and the article comprising a plurality of discrete portions of aerosol generating material.

FIGS. 2A, 2B, and 2C are a variety of views from different angles of the aerosol provision system of FIG. 1.

FIG. 3 is cross-sectional, top-down view of the heating elements of the aerosol provision system of FIG. 1.

FIG. 4 is a cross-sectional schematic representation of an aerosol provision system according to a non-limiting embodiment of the disclosure, comprising an aerosol provision device and an aerosol generating article, the device comprising a plurality of induction work coils and the article comprising a plurality of discrete portions of aerosol generating material and corresponding susceptor portions.

FIGS. 5A, 5B, and 5C are a variety of views from different angles of the aerosol provision system of FIG. 4.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

Reference to “dry weight percent” or “dry weight basis” refers to weight on the basis of dry ingredients (i.e., all ingredients except water). All weight percent values herein are dry weight percent unless otherwise indicated.

The terms ‘upstream’ and ‘downstream’ used herein are relative terms defined in relation to the direction of mainstream aerosol drawn though an article or device in use.

Unless otherwise defined herein, by “substantially free” it is meant that the noted component (e.g., nicotine or a tobacco material) has not been intentionally added, beyond trace amounts that may be present e.g., as an impurity in another component, or small amounts which may be present in certain flavor packages. For example, some embodiments can have less than 0.01% by weight of the noted component, or less than 0.001%, or even 0% by weight of the noted component, based on the total weight of the aerosol generating material. In some embodiments, the aerosol generating material is completely free of the noted component (i.e., having 0% or as having an amount below the limit of detection).

### Consumable

The present disclosure is generally directed to a consumable, which may be used in an aerosol provision system, such as a non-combustible aerosol provision system. A consumable is an article comprising or consisting of aerosol generating material, part or all of which is intended to be consumed during use by a user. A consumable may comprise one or more other components, such as an aerosol generating material storage area, an aerosol generating material transfer component, an aerosol generation area, a housing, a wrapper, a mouthpiece, and/or a filter. A consumable may also comprise an aerosol generator, such as a heater, that emits heat to cause the aerosol generating material to generate aerosol in use. The heater may, for example, comprise combustible material, a material heatable by electrical conduction, or a susceptor. The consumables are sometimes referred to as “articles” or “aerosol generating articles” throughout the disclosure.

In particular, the consumable comprises one or more discrete portions of aerosol generating material, the aerosol generating material comprising an aerosol former, a binder and an active agent. An advantage of the consumable of the present disclosure is that there is a controlled and defined amount of active agent in each of the discrete portions of aerosol generating material. This enables the amount of active agent aerosolized during use to be controlled, such that an accurate dose of the active can be delivered to a user.

In this regard, during use each of the discrete portions of aerosol generating material is generally heated separately and independently, with a set number of portions (often just one) being heated to or above an aerosol generating temperature at any one time. Only the portion or portions of material which is or are being heated to or above an aerosol generating temperature will release aerosol, and consequently the amount of active agent being aerosolized at any time can be controlled. Conversely, if the material was not in the form of discrete portions (but rather a continuous or bulk solid or liquid), and/or if the amount of active agent in each of the discrete portions was not controlled, the amount of active agent being aerosolized at any one time could not be as accurately controlled or determined. In this case, the amount of active agent being delivered to a user could not be controlled or determined.

The accurate control of the amount of active agent being aerosolized is particularly important since it is usually desired and/or required that the amount of active agent being delivered to a user is controlled or known, due to health and/or regulatory reasons. Control of the price of the overall consumable may be another advantage to having a controlled and determined amount of active agent in each discrete portion.

The consumable, the components thereof, and articles and systems comprising the consumable are described further herein below.

### Aerosol Generating Material

In one aspect is provided a consumable comprising one or more discrete portions of aerosol generating material, the aerosol generating material comprising an aerosol former, a binder, and an active agent.

### Aerosol Former

The aerosol generating material as disclosed herein comprises an aerosol former. In this context, an “aerosol former” or “aerosol forming material” is an agent that promotes the generation of an aerosol. An aerosol former may promote the generation of an aerosol by promoting an initial vaporization and/or the condensation of a gas to an inhalable solid and/or liquid aerosol. In general, any suitable aerosol former may be included in the aerosol generating material of the disclosure.

In some embodiments, the aerosol generating material comprises from about 1 wt %, 5 wt %, 10 wt %, 12 wt % or 13 wt % to about 18 wt %, 20 wt %, 25 wt %, 30 wt %, 35 wt %, 45 wt %, 55 wt %, 65 wt %, 75 wt % or 80 wt % of an aerosol former (all calculated on a dry weight basis). In some embodiments, the aerosol generating material comprises 1-80 wt %, 1-50 wt %, 5-35 wt %, 10-25 wt %, 12-20 wt % or 13-18 wt % of an aerosol former (all calculated on a dry weight basis).

The particular choice of aerosol former(s) may depend on factors such as the method of aerosol formation, the appearance and volume of the aerosol, the desired density of the aerosol, and the like. Suitable aerosol formers include, but are not limited to: a polyol such as sorbitol, glycerol, and glycols like propylene glycol or triethylene glycol; a non-polyol such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate. In some embodiments, the aerosol former may be glycerol, propylene glycol, or a mixture of glycerol and propylene glycol.

In some embodiments, the aerosol former may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

In some embodiments, the aerosol former comprises one or more compound selected from erythritol, propylene glycol, glycerol, triacetin, sorbitol and xylitol. In some embodiments, the aerosol former material comprises, consists essentially of, or consists of, glycerol.

In some embodiments, the aerosol former comprises a mixture of glycerol and propylene glycol in a weight ratio of glycerol to propylene glycol of from about 3:1 to about 1:3, from about 2:1 to about 1:2, from about 1.5:1 to about 1:1.5, from about 55:45 to about 45:55, or about 45:55.

### Binder

The aerosol generating material as disclosed herein comprises a binder. The binder generally comprises an alginate, pectin, starch (and derivatives), cellulose (and derivatives), gum, silica or silicone compounds, clay, polyvinyl alcohol, or combinations thereof. In some embodiments, the binder comprises (or is) a hydrocolloid. In some embodiments, the binder comprises (or is) one or more compounds selected from the group consisting of alginates, pectins, starches (and derivatives), celluloses (and derivatives, such as such as methylcellulose, hydroxypropyl cellulose, and carboxymethyl cellulose (CMC)), gums, silica or silicones compounds, clays, polyvinyl alcohol and combinations thereof. For example, in some embodiments, the binder comprises (or is) one or more of alginates, pectins, hydroxyethyl cellulose, hydroxypropyl cellulose, carboxymethylcellulose, pullulan, xanthan gum, guar gum, carrageenan, agarose, acacia gum, fumed silica, PDMS, sodium silicate, kaolin and polyvinyl alcohol.

In some embodiments, the binder is a cellulosic binder, which may be selected from the group consisting of: hydroxymethyl cellulose, hydroxyethyl cellulose, hydroxypropyl cellulose, carboxymethylcellulose (CMC), hydroxypropyl methylcellulose (HPMC), methyl cellulose, ethyl cellulose, cellulose acetate (CA), cellulose acetate butyrate (CAB), cellulose acetate propionate (CAP) and combinations thereof.

In some embodiments, the binder comprises (or is) a non-cellulosic binder, which may be selected from the group consisting of agar, xanthan gum, gum Arabic, guar gum, locust bean gum, pectin, carrageenan, starch, alginate, and combinations thereof. In some embodiments, the non-cellulose binder is alginate. In some embodiments, the binder comprises (or is) one or more of hydroxyethyl cellulose, hydroxypropyl cellulose, hydroxypropyl methylcellulose (HPMC), carboxymethylcellulose, alginate, pectin, guar gum, and acacia gum.

In some embodiments, the binder comprises alginate and/or pectin.

In some embodiments, the binder comprises, consists essentially of, or consists of alginate and pectin.

In some embodiments, the binder comprises, consists essentially of, or consists of one or more carboxymethylcellulose, alginate, and pectin.

In some embodiments, the aerosol generating material comprises from about 0.5 wt % to about 60 wt % of a binder, such as from about 5 wt % to about 50 wt %, from about 10 wt % to about 35 wt %, from about 15 wt % to about 30 wt %, or from about 15 wt % to about 25 wt %.

### Filler

In some embodiments, the aerosol generating material comprises a filler. Use of a filler may help to reduce tackiness of the aerosol generating material, for example if high levels of aerosol-former material are present.

In some embodiments, the aerosol generating material comprises less than about 50 wt % of a filler, such as from about 1 wt % to 50 wt %, or 5 wt % to 40 wt %, or 5 wt % to 30 wt %, or 10 wt % to 20 wt %.

In some embodiments, the aerosol generating material comprises from about 1 wt %, 5 wt %, 10 wt %, 18 wt % or 20 wt % to about 50 wt %, 45 wt %, 40 wt %, 35 wt % or 30 wt % of filler (all calculated on a dry weight basis). For example, the aerosol generating material may comprise 5-45 wt %, 10-40 wt %, 18-35 wt % or 20-30 wt % of filler (all calculated on a dry weight basis). These amounts represent the total amount of filler(s) in the aerosol generating material.

In some embodiments, the aerosol generating material comprises less than 20 wt %, suitably less than 10 wt % or less than 5 wt % of a filler. In some cases, the aerosol generating material comprises less than 1 wt % of a filler, and in some cases the aerosol generating material comprises no filler. In some embodiments, a low quantity of filler (e.g., less than about 20% by weight) may be advantageous in reducing the thermal mass which is subject to heating, thereby resulting in more rapid heating of the aerosol generating material and more rapid evolution of aerosol.

The filler, if present, may comprise one or more inorganic filler materials, such as calcium carbonate, chitosan, perlite, vermiculite, diatomaceous earth, colloidal silica, magnesium oxide, magnesium sulphate, magnesium carbonate, and suitable inorganic sorbents, such as molecular sieves. The filler may comprise one or more organic filler materials such as wood pulp; tobacco pulp; hemp fibre; starch and starch derivatives, such as maltodextrin; and cellulose and cellulose derivatives, such as ground cellulose, microcrystalline cellulose and nanocrystalline cellulose. In particular cases, the aerosol generating material comprises no calcium carbonate such as chalk.

In some embodiments, the filler is fibrous. For example, the filler may be a fibrous organic filler material such as wood pulp, hemp fiber, cellulose or cellulose derivatives, such as microcrystalline cellulose (MCC) and/or nanocrystalline cellulose. In some cases, the filler comprises maltodextrin or microcrystalline cellulose (MCC).

As would be well understood by the skilled person, microcrystalline cellulose may be formed by depolymerizing cellulose by a chemical process (e.g. using an acid or enzyme). One example method for forming microcrystalline cellulose involves acid hydrolysis of cellulose, using an acid such as HCl. The cellulose produced after this treatment is crystalline (i.e. no amorphous regions remain). Suitable methods and conditions for forming microcrystalline cellulose are well-known in the art.

In some cases, the filler comprises, consists essentially of or consists of wood pulp, calcium carbonate and combinations thereof.

In some cases, the filler comprises, consists essentially of or consists of wood pulp and calcium carbonate.

In some cases, the filler comprises, consists essentially of or consists of wood pulp. The aerosol generating material may comprise about 1 wt %, 5 wt %, 10 wt %, 12 wt % or 13 wt % to about 15 wt %, 17 wt % or 20 wt % of wood pulp (all calculated on a dry weight basis).

The aerosol generating material may comprise from about 10 wt %, 20 wt %, 30 wt %, 35 wt %, 40 wt % or 45 wt % to about 55 wt %, 60 wt %, 65 wt % or 70 wt % of calcium carbonate (all calculated on a dry weight basis).

### Active Agent

### Substituted 3-(1-methylpyrrolidin-2-yl)pyridine

In some embodiments, the aerosol generating material of the disclosure comprises an active agent, including, but not limited to, a substituted 3-(1-methylpyrrolidin-2-yl)pyridine. The active agent may further include components such as cannabinoids, terpenes, vitamins, melatonin, caffeine, tobacco extract, or combinations thereof. The various components are described further herein below.

As used herein, the term “substituted 3-(1-methylpyrrolidin-2-yl)pyridine” refers to a compound having a 3-(1-pyrrolidin-2-yl)pyridine) scaffold and bearing one or more non-hydrogen substituents on the pyridine ring.

In some embodiments, the substituted 3-(1-methylpyrrolidin-2-yl)pyridine has a structure according to Formula I:

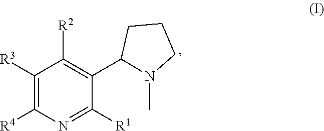

wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen.

Substituted 3-(1-methylpyrrolidin-2-yl)pyridines with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34(1): 31-40.

In some embodiments, R¹, R², and R⁴ are each H, and R³ is a non-hydrogen substituent. In some embodiments, R³ is optionally substituted C₁-C₆ alkyl, F, Cl, Br, OMe, OEt, or CN. In some embodiments, R³ is Me or Et. n some embodiments, R³ is F.

In some embodiments, R¹, R², and R³ are each H, and R⁴ is a non-hydrogen substituent.

In some embodiments, R⁴ is optionally substituted C₁-C₆ alkyl, F, Cl, Br, OMe, OEt, or CN.

In some embodiments, R¹, R², and R³ are each H, and R⁴ is optionally substituted C₁-C₆ alkyl, F, Cl, Br, OCH₃, OEt, or CN.

In some embodiments, R¹, R², and R³ are each H, and R⁴ is C₁-C₃ alkyl.

In some embodiments, R¹, R², and R³ are each H, and R⁴ is Me. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; 76^{th} TSRC Conference 2023, Norfolk, VA, USA).

In some embodiments, the substituted 3-(1-methylpyrrolidin-2-yl)pyridine has a structure according to Formula II:

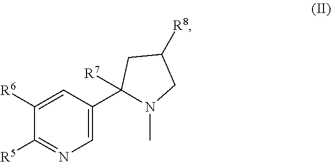

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C₁-C₃ alkyl; and at least one of R⁷ and R⁸ is not hydrogen.

Certain substituted 3-(1-methylpyrrolidin-2-yl)pyridines with various R⁵, R⁶, R⁷, and R⁸ substituents have been reported previously. See for example, U.S. Pat. Nos. 4,321,387, 4,155,909; 5,015,741, 5,138,062, and 5,703,100, each of which is incorporated by reference herein and describe example substituted 3-(1-methylpyrrolidin-2-yl)pyridines, their synthesis, and pharmacological properties. Certain substituted 3-(1-methylpyrrolidin-2-yl)pyridines and their pharmacological profiles have also been disclosed in Wang et al., Drug Development Research 1998, Volume 45, Issue 1, Pages 10-16; Dukat et al. European Journal of Medicinal Chemistry 1999, 34(1): 31-40; Lin et al., J. Med. Chem. (1994), 37, 3542-3553.

In some embodiments, R⁵ and R⁶ are H; R⁷ is CH₃; and R⁸ is H. In such embodiments, the compound of Formula II may be referred to as 3-(1,2-dimethylpyrrolidin-2-yl)pyridine, or 2′-methyl-5-(1-methylpyrrolidin-2-yl)pyridine, and has a structure:

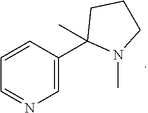

The compound 3-(1,2-dimethylpyrrolidin-2-yl)pyridine is known in the literature, and has a Chemical Abstracts Registry Number of 220650-38-4. The synthesis of this compound has been reported in Rouchaud et al., J Het Chem 2012, 49(1), 161-166; Wang et al., Drug Dev Res 1998, 45(1), 10-16; Secor et al., Tetrahedron Lett. (1981), 22(33), 3151-3154; US Patent Application Publication No. 2013/0157995; PCT Application Publication No. WO2012/031220; and U.S. Pat. No. 9,440,948, each of which are incorporated by reference herein with respect to the synthesis of 3-(1,2-dimethylpyrrolidin-2-yl)pyridine.

In some embodiments, R⁵ and R⁶ are H; R⁷ is H; and R⁸ is CH₃. In such embodiments, the compound of Formula II may be referred to as 3-(1,4-dimethylpyrrolidin-2-yl)pyridine, or 4′-methyl-5-(1-methylpyrrolidin-2-yl)pyridine, and has a structure:

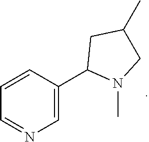

The compound 3-(1,4-dimethylpyrrolidin-2-yl)pyridine is known in the literature, has a Chemical Abstracts Registry Number of 74805-00-8, and is commercially available from, for example, Enamine Stock Building Blocks and Aurora Building Blocks. The synthesis of this compound has been reported in U.S. Pat. No. 9,440,948; and EP Patent No. 559495, each of which are incorporated by reference herein with respect to the synthesis of 3-(1,4-dimethylpyrrolidin-2-yl)pyridine.

In some embodiments, R⁵ is CH₃; R⁶ is H; R⁷ is CH₃; and R⁸ is H. In such embodiments, the compound of Formula II may be referred to as 5-(1,2-dimethylpyrrolidin-2-yl)-2-methylpyridine, and has a structure:

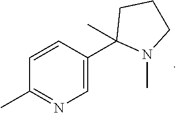

The compound 5-(1,2-dimethylpyrrolidin-2-yl)-2-methylpyridine may be readily synthesized according to known reactions. For example, commercially available 2-methyl-5-(2-methylpyrrolidin-2-yl)pyridine (Chemical Abstracts Registry Number of 1528955-30-7; Aurora Building Blocks, Adlab Chemicals Building Blocks) can be N-methylated with formaldehyde and formic acid, or alternatively with formaldehyde and a reducing agent such as sodium cyanoborohydride to afford 5-(1,2-dimethylpyrrolidin-2-yl)-2-methylpyridine. Alternatively, 5-(1,2-dimethylpyrrolidin-2-yl)-2-methylpyridine may be synthesized by lithiation of 2-methyl-5-bromopyridine, reaction of the lithiated pyridine with N-methylpyrrolidone, and addition of methyl lithium to the perchlorate salt of the resulting imine. This reaction sequence is shown below in Scheme 1.

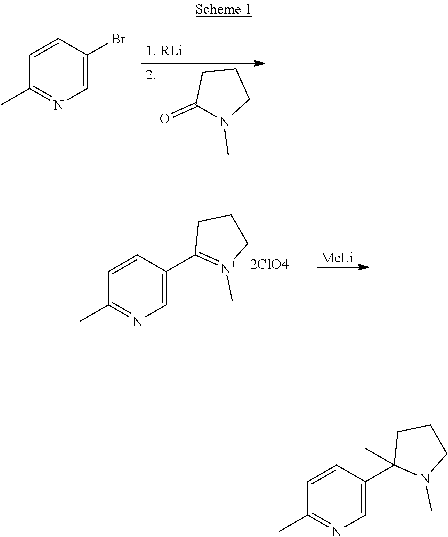

In some embodiments, R⁵ is H or CH₃, R⁶ is H, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from:

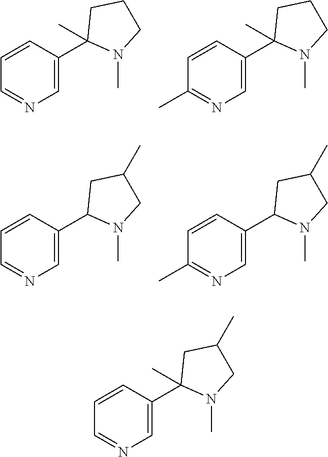

In some embodiments, R⁵ is H, R⁶ is F, CH₃, or OCH₃, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from:

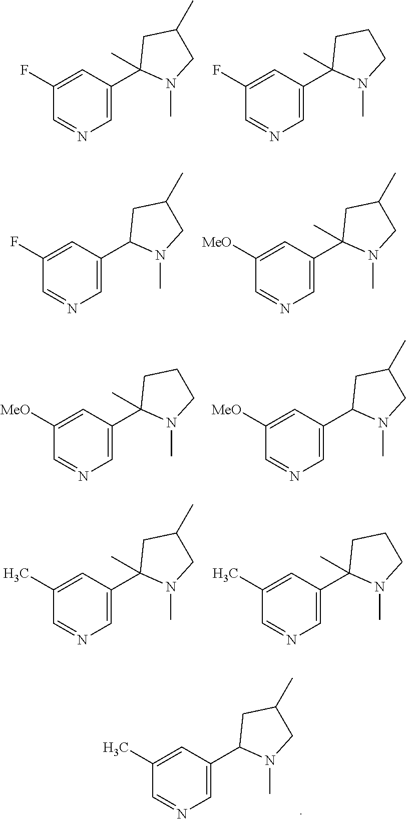

A substituted 3-(1-methylpyrrolidin-2-yl)pyridine and bearing one or more substituents on the pyrrolidine ring as described herein may be present as a single enantiomer or as a mixture of enantiomers. In some embodiments, the substituted 3-(1-methylpyrrolidin-2-yl)pyridine bearing one or more substituents on the pyrrolidine ring is present in racemic form, meaning there are equal amounts of (R)- and (S)-enantiomers present. In some embodiments, the aerosol generating material comprises unequal amounts of (R)- and (S)-enantiomer (i.e., is enriched in either the (R)- or (S)-enantiomer. In some embodiments, the aerosol generating material predominantly comprises the substituted 3-(1-methylpyrrolidin-2-yl)pyridine bearing one or more substituents on the pyrrolidine ring in the (R)-configuration, for example, about 90% or more of the total quantity of substituted 3-(1-methylpyrrolidin-2-yl)pyridine bearing one or more substituents on the pyrrolidine ring present is in the (R)-configuration. In some embodiments, the aerosol generating material predominantly comprises the substituted 3-(1-methylpyrrolidin-2-yl)pyridine bearing one or more substituents on the pyrrolidine ring in the (S)-configuration, for example, about 90% or more of the total quantity of substituted 3-(1-methylpyrrolidin-2-yl)pyridine present is in the (S)-configuration. In some embodiments, the aerosol generating material comprises 95% or more of the (S)-configuration of the substituted 3-(1-methylpyrrolidin-2-yl)pyridine, based on the total amount of substituted 3-(1-methylpyrrolidin-2-yl)pyridine present.

In some embodiments, the substituted 3-(1-methylpyrrolidin-2-yl)pyridine bearing one or more substituents on the pyrrolidine ring is non-racemic, and has one of the following structures:

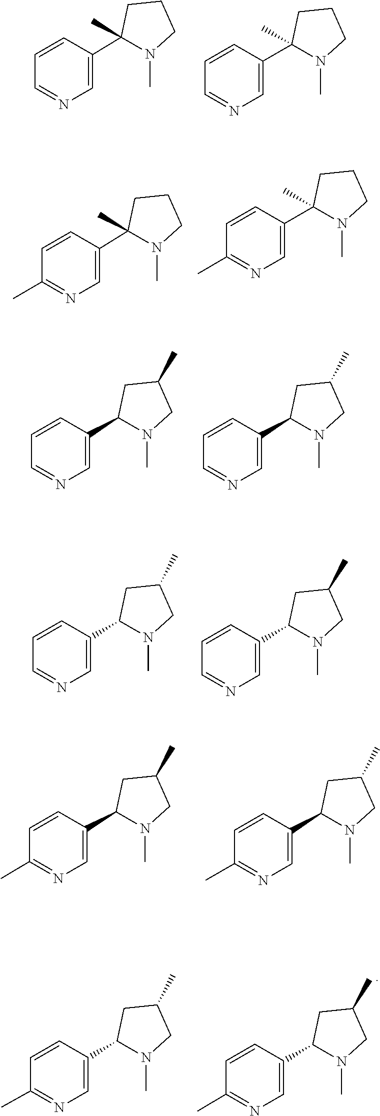

In some embodiments, the substituted 3-(1-methylpyrrolidin-2-yl)pyridine bearing one or more substituents on the pyrrolidine ring is non-racemic, and has a structure selected from:

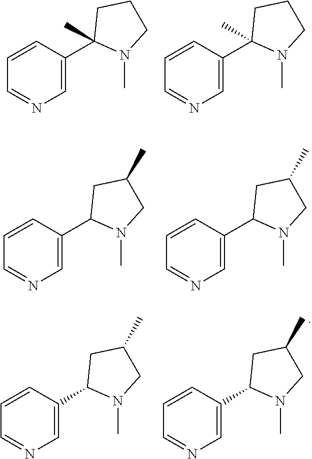

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of various substituted 3-(1-methylpyrrolidin-2-yl)pyridines such as those described herein has been reported in, for example, Lin et al., J. Med. Chem., 1994, 37, 3542-3553; Dukat et al. European Journal of Medicinal Chemistry, 31(11), 1996, 875-888; U.S. Pat. No. 9,440,948; Wang et al., Drug Dev Res 1998, 45(1), 10-16 (each of which is incorporated herein by reference), among many others. Generally, small substituents such as methyl groups are well tolerated at the 2′ or 4′ positions of the nicotine pyrrolidine ring, and small substituents such as alkyl, halogen, alkoxy, and the like are well tolerated at the 5 or 6 position of the nicotine pyridine ring. For example, 3-(1-methylpyrrolidin-2-yl)pyridines bearing a methyl substituent at the 2′ or 4′ position are equipotent or even more potent than nicotine with respect to binding affinity to the nicotinic acetylcholine receptor, and are expected to preserve the pharmacological effects of nicotine in vivo. See, for example U.S. Pat. No. 5,278,176, Lin et al., J. Med. Chem., 1994, 37, 3542-3553, and Wang et al., Drug Dev Res 1998, 45(1), 10-16.

Without wishing to be bound by any particular theory, it is believed that certain substitutions for hydrogen on the pyridine ring and/or pyrrolidine rings of the substituted 3-(1-methylpyrrolidin-2-yl)pyridine compound preserve the general pharmacological profile and physiological effects of nicotine while offering the potential for one or more of greater potency, longer half-life, reduced product consumption, more rapid and/or complete absorption, greater bioavailability, and the like. Particularly, it is believed that in some embodiments, substituted 3-(1-methylpyrrolidin-2-yl)pyridines of the disclosure are readily absorbed through membranes such as pulmonary alveoli, oral mucosa, and the like by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I or II has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I or II has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I or II has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine is 2-methyl-5-(1-methylpyrrolidin-2-yl)pyridine.

The quantity of substituted 3-(1-methylpyrrolidin-2-yl)pyridine (e.g., 2-methyl-5-(1-methylpyrrolidin-2-yl)pyridine) present in each discrete portion of the aerosol generating material may vary. In some embodiments, each discrete portion comprises from about 001 to about 5 mg of the substituted 3-(1-methylpyrrolidin-2-yl)pyridine.

In some embodiments, the total amount of aerosol generating material present comprises from about 0.01 to about 5 wt % of the substituted 3-(1-methylpyrrolidin-2-yl)pyridine. In some embodiments, the aerosol generating material comprises 2-methyl-5-(1-methylpyrrolidin-2-yl)pyridine in an amount from about 0.01 to about 1% by weight, such as from about 0.1 to about 0.75% by weight, based on the total weight of the aerosol generating material.

The substituted 3-(1-methylpyrrolidin-2-yl)pyridine (e.g., 2-methyl-5-(1-methylpyrrolidin-2-yl)pyridine) may be present in the aerosol generating material as the free base, as a salt with a suitable acid, or a combination thereof. Each of these forms is described further herein below.

As described above, aerosol generating materials of the disclosure do not contain nicotine and do not contain any compounds obtained by chemical reactions utilizing nicotine as a starting material.

### 3-(azetidin-2-yl)pyridines and 3-(azetidin-2-ylmethoxy)pyridines

In some embodiments, the aerosol generating material of the disclosure comprises an optionally substituted 3-(azetidin-2-yl)pyridine or an optionally substituted 3-(azetidin-2-ylmethoxy)pyridine. As used herein, the term “substituted 3-(azetidin-2-yl)pyridine” refers to a compound having a 3-(azetidin-2-yl)pyridine scaffold and bearing one or more non-hydrogen substituents on the azetidine ring, and optionally on the pyridine ring. As used herein, the term “substituted 3-(azetidin-2-ylmethoxy)pyridine” refers to a compound having a 3-(azetidin-2-ylmethoxy)pyridine scaffold and bearing one or more non-hydrogen substituents on the azetidine ring, and optionally on the pyridine ring.

In some embodiments, the 3-(azetidin-2-yl)pyridine or 3-(azetidin-2-ylmethoxy)pyridine has a structure according to Formula III:

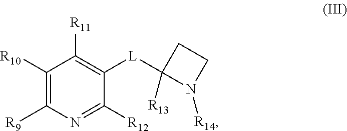

wherein:
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

In some embodiments, L is a bond.

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
   R¹³ and R¹⁴ are both CH₃;
R¹³ is H and R¹⁴ is CH₃; or
R¹³ is CH₃ and R¹⁴ is H.

In some embodiments, the 3-(azetidin-2-yl) pyridine is 3-(azetidin-2-yl)pyridine, and has a structure:

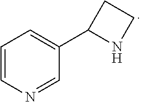

The compound 3-(azetidin-2-yl)pyridine is known in the literature. The synthesis of this compound has been reported in JOC 1979, 44(18), 3136; Med Chem Res (1993) 2:552-5633; in International Patent Application Publication No. WO2012/031220, and in U.S. Pat. Nos. 4,163,855 and 4,163,856, all of which are incorporated herein in their entireties.

In some embodiments, the 3-(azetidin-2-yl) pyridine is 3-(1-methylazetidin-2-yl)pyridine, having the structure:

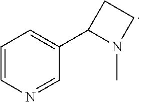

The compound 3-(1-methylazetidin-2-yl)pyridine is known in the literature. The synthesis of this compound has been reported in International Patent Application Publication No. WO2012/031220, previously incorporated by reference herein.

In some embodiments, the 3-(azetidin-2-yl) pyridine has a structure selected from the group consisting of:

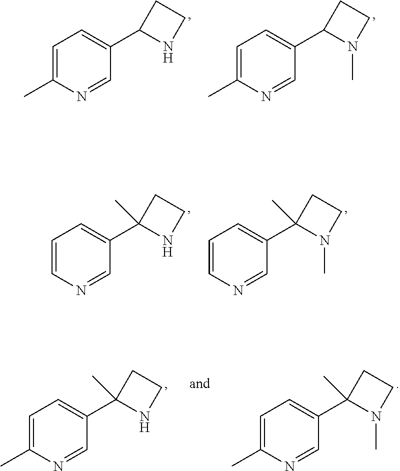

Such compounds are either known, or may be readily prepared according to adaptations of methods utilized for preparation of related 3-(azetidin-2-yl) pyridines and 3-(1-methylpyrrolidin-2-yl)pyridines described herein above. See, e.g., U.S. Pat. No. 4,163,855, previously incorporated by reference herein. The compound 5-(2-azetidinyl)-2-methylpyridine is known in the literature and has a Chemical Abstracts Registry (CAS) Number of 1270467-65-6, and the R- and S-enantiomers have CAS numbers 1213081-15-2 and 1212969-96-4, respectively.

In some embodiments, the aerosol generating material comprises a 3-(azetidin-2-ylmethoxy)pyridine (i.e., L is —OCH₂—*).

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-ylmethoxy)pyridine has a structure selected from the group consisting of:

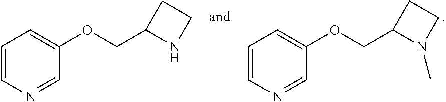

These compounds are known in the literature. The synthesis of these compounds has been reported in International Patent Application Publication No. WO2012/031220, previously incorporated by reference herein.

In some embodiments, the 3-(azetidin-2-ylmethoxy)pyridine has a structure selected from the group consisting of:

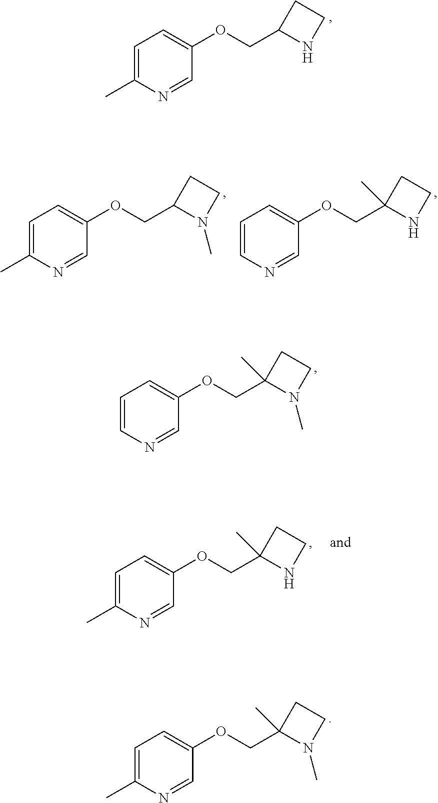

Such compounds are either known, or may be readily prepared according to adaptations of methods utilized for preparation of related the 3-(azetidin-2-ylmethoxy)pyridine, and/or the 3-(azetidin-2-yl)pyridines and substituted 3-(1-methylpyrrolidin-2-yl)pyridines described herein above.

An optionally substituted 3-(azetidin-2-yl)pyridine or optionally substituted 3-(azetidin-2-ylmethoxy)pyridine as described herein may be present as a single enantiomer or as a mixture of enantiomers. In some embodiments, the optionally substituted 3-(azetidin-2-yl)pyridine or optionally substituted 3-(azetidin-2-ylmethoxy)pyridine is present in racemic form, meaning there are equal amounts of (R)- and (S)-enantiomers present. In some embodiments, the aerosol generating material comprises unequal amounts of (R)- and (S)-enantiomer (i.e., is enriched in either the (R)- or (S)-enantiomer). In some embodiments, the aerosol generating material predominantly comprises the optionally substituted 3-(azetidin-2-yl)pyridine or optionally substituted 3-(azetidin-2-ylmethoxy)pyridine in the (R)-configuration, for example, about 90% or more of the total quantity of optionally substituted 3-(azetidin-2-yl)pyridine or optionally substituted 3-(azetidin-2-ylmethoxy)pyridine present is in the (R)-configuration. In some embodiments, the aerosol generating material predominantly comprises the optionally substituted 3-(azetidin-2-yl)pyridine or optionally substituted 3-(azetidin-2-ylmethoxy)pyridine in the (S)-configuration, for example, about 90% or more of the total quantity of optionally substituted 3-(azetidin-2-yl)pyridine or optionally substituted 3-(azetidin-2-ylmethoxy)pyridine present is in the (S)-configuration. In some embodiments, the aerosol generating material comprises 95% or more of the (S)-configuration of the optionally substituted 3-(azetidin-2-yl)pyridine or optionally substituted 3-(azetidin-2-ylmethoxy)pyridine, based on the total amount of optionally substituted 3-(azetidin-2-yl)pyridine or optionally substituted 3-(azetidin-2-ylmethoxy)pyridine present.

In some embodiments, the optionally substituted 3-(azetidin-2-yl)pyridine is non-racemic, and has one of the following structures:

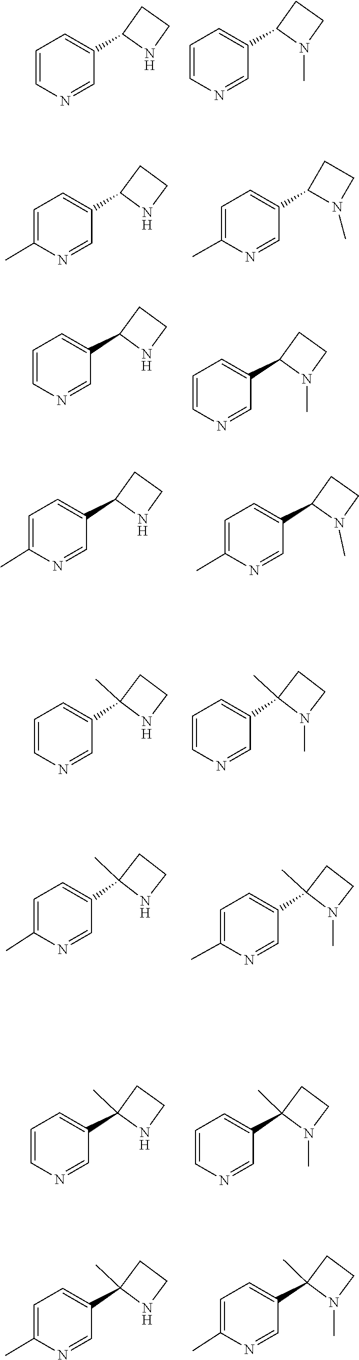

In some embodiments, the optionally substituted 3-(azetidin-2-ylmethoxy)pyridine is non-racemic, and has one of the following structures:

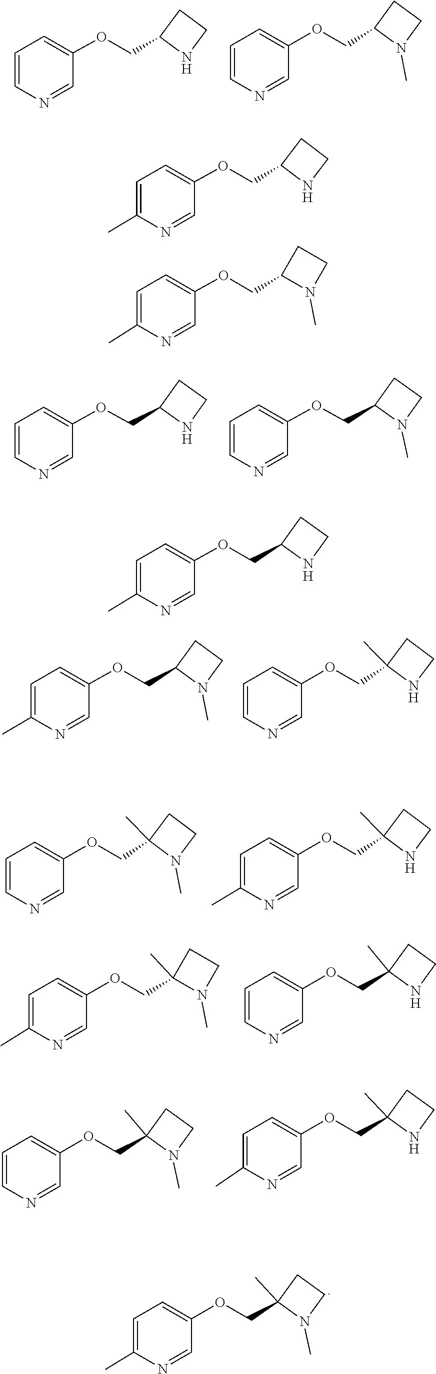

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of certain 3-(azetidin-2-yl)pyridines and 3-(azetidin-2-ylmethoxy)pyridines has been previously disclosed, for example, in the references cited herein with respect to synthesis of such compounds. Generally, these compounds exhibit high affinity for one or more subtypes of nicotinic acetylcholine receptors, particularly the a4b2 subtype. The overall pharmacological profiles have been shown to be or are expected to be comparable to that of nicotine.

The quantity of optionally substituted 3-(azetidin-2-yl)pyridine or 3-(azetidin-2-ylmethoxy)pyridine present in the aerosol generating material may vary. Typically, the optionally substituted 3-(azetidin-2-yl)pyridine or 3-(azetidin-2-ylmethoxy)pyridine, calculated as the free base, is present in a concentration of at least about 0.001% by weight of the aerosol generating material, such as in a range from about 0.01% to about 10%. In some embodiments, the optionally substituted 3-(azetidin-2-yl)pyridine or 3-(azetidin-2-ylmethoxy)pyridine is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the aerosol generating material. In some embodiments, the optionally substituted 3-(azetidin-2-yl)pyridine or 3-(azetidin-2-ylmethoxy)pyridine is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the aerosol generating material. One of skill in the art will recognize that the amount of any particular optionally substituted 3-(azetidin-2-yl)pyridine or 3-(azetidin-2-ylmethoxy)pyridine present in the aerosol generating material may vary based on the potency of the compound, the aerosol generating material matrix, and the desired physiological effect for the aerosol generating material.

In some embodiments, the amount of optionally substituted 3-(azetidin-2-yl)pyridine or 3-(azetidin-2-ylmethoxy)pyridine in the aerosol generating material is determined by potency relative to nicotine. For example, in some embodiments, the amount of optionally substituted 3-(azetidin-2-yl)pyridine or 3-(azetidin-2-ylmethoxy)pyridine is based on the potency factor as described above for substituted 3-(1-methylpyrrolidin-2-yl)pyridines. In some embodiments, an optionally substituted 3-(azetidin-2-yl)pyridine or 3-(azetidin-2-ylmethoxy)pyridine of the disclosure has a potency factor from about 0.1 to about 30, such as from about 2 to about 30.

In some embodiments, the amount of optionally substituted 3-(azetidin-2-yl)pyridine or 3-(azetidin-2-ylmethoxy)pyridine in the aerosol generating material is 1 nicotine equivalent. Accordingly, in some embodiments, 1 nicotine equivalent of optionally substituted 3-(azetidin-2-yl)pyridine or 3-(azetidin-2-ylmethoxy)pyridine is an amount by weight from about 10 to about 0.03 times that of nicotine. In some embodiments, 1 nicotine equivalent of optionally substituted 3-(azetidin-2-yl)pyridine or 3-(azetidin-2-ylmethoxy)pyridine is an amount by weight from about 0.5 to about 0.03 times that of nicotine.

For example, a product comprising 2 mg of nicotine, when the nicotine is replaced by an optionally substituted 3-(azetidin-2-yl)pyridine or 3-(azetidin-2-ylmethoxy)pyridine of the disclosure, may include from about 0.06 mg to about 1 mg of the optionally substituted 3-(azetidin-2-yl)pyridine or 3-(azetidin-2-ylmethoxy)pyridine. Similarly, a product comprising 20 mg of nicotine, when the nicotine is replaced by an optionally substituted 3-(azetidin-2-yl)pyridine or 3-(azetidin-2-ylmethoxy)pyridine, may contain from about 0.6 mg to about 100 mg of the optionally substituted 3-(azetidin-2-yl)pyridine or 3-(azetidin-2-ylmethoxy)pyridine.

In some embodiments, the optionally substituted 3-(azetidin-2-yl)pyridine has the structure:

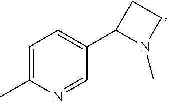

having a potency factor of about 30, meaning that in some embodiments, the amount of optionally substituted 3-(azetidin-2-yl)pyridine present may be about 3% of the amount of nicotine required to achieve the same effect.

In some embodiments, the optionally substituted 3-(azetidin-2-ylmethoxy)pyridine has the structure:

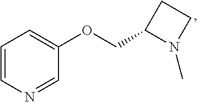

having a potency factor of about 3, meaning that in some embodiments, the amount of 3-(azetidin-2-ylmethoxy)pyridine present may be about 33% of the amount of nicotine required to achieve the same effect.

The optionally substituted 3-(azetidin-2-yl)pyridine or 3-(azetidin-2-ylmethoxy)pyridine may be present in the aerosol generating material as the free base, as a salt with a suitable acid, or in the form of an ion pair with an organic acid. Each of these forms is described further herein below.

### Other Active Agents

In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine of the present disclosure is replaced with, or combined with, other active agents that provide the same general pharmacological profile and/or physiological effects of nicotine. Certain of these active agents may be equipotent or even more potent than nicotine with respect to binding affinity to the nicotinic acetylcholine receptor and are expected to preserve the pharmacological effects of nicotine in vivo. Without wishing to be bound by any particular theory, in some embodiments, these compounds are believed to provide the general pharmacological profile and physiological effects of nicotine while offering the potential for one or more of greater potency, reduced product consumption, more rapid and/or complete absorption, and the like.

Example active agents of this type include, without limitation, cytisine, varenicline, acetylcholine, choline, epibatidine, lobeline, analogs thereof, or combinations thereof. Suitable analogs include any of the above-noted compounds having one or more substituents on any of the carbon atoms thereof, with example substituents including alkyl (e.g., C₁-C₃ alkyl), alkoxy, cycloalkyl, alkenyl, alkynyl, aryl, alkylaryl, amino, halogen, and cyano.

In some embodiments, the other active agent is cytisine or an analog thereof. Cytisine is a naturally occurring alkaloid present in certain plant genera, such as Laburnum and Cytisus of the family Fabaceae. Cytisine (CAS Registry No. 485-35-8) has the structure:

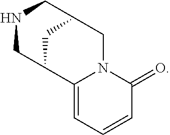

Cytisine is commercially available and has been utilized in post-Soviet states for more than 40 years as an aid to smoking cessation under the brand name Tabex (Sopharma AD). Cytisine is a partial agonist of the α₄β₂ nicotinic acetylcholine receptor.

In some embodiments, the other active agent is varenicline or an analog thereof. Varenicline is commercially available as Chantix® (Pfizer) and is a medication used as an aid for smoking cessation. Varenicline (CAS Registry No. 249296-44-4) has the structure:

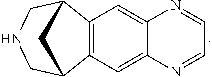 [SMILES: c1cnc2cc3c(cc2n1)[C@@H]1CNC[C@H]3C1]

Like cytisine, varenicline is a partial agonist of the α₄β₂ nicotinic acetylcholine receptor.

The quantity of the other active agent present in the aerosol generating material may vary. Typically, the other active agent, calculated as the free base, is present in a concentration of at least about 0.001% by weight of the aerosol generating material, such as in a range from about 0.01% to about 10%. In some embodiments, the other active agent is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the aerosol generating material. In some embodiments, the other active agent is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the aerosol generating material. One of skill in the art will recognize that the amount of any particular other active agent present in the aerosol generating material may vary based on the potency of the compound, the aerosol generating material matrix, and the desired physiological effect for the aerosol generating material.

The other active agent may be present in the aerosol generating material as the free base, as a salt with a suitable acid, or in the form of an ion pair with an organic acid. Each of these forms is described further herein below.

### Free Base

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent exhibits sufficient stability, aqueous solubility, and oral bioavailability such that the free base is suitable for inclusion in the aerosol generating material. Accordingly, in some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine or other active agent is present substantially or completely as the free base. In such embodiments, one of skill in the art will recognize that the aerosol generating material is substantially free of acidic components. By “substantially free” it is meant that no acidic component (e.g., inorganic acid, organic acid, or acids capable of salt has been intentionally added, beyond trace amounts that may be present e.g., as an impurity in another component, or small amounts which may be present in certain flavor packages. For example, some embodiments can have less than 0.001% by weight of any acid component, or less than 0.0001%, or even 0% by weight of any acid component, based on the total weight of the aerosol generating material. In some embodiments, the aerosol generating material is completely free of any acid component (i.e., having 0% or having an amount below the limit of detection). In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the free base form and is adsorbed in a carrier such as a microcrystalline cellulose material to form an adsorption complex.

### Salt

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent can be employed in the form of a salt. A “salt” of such compounds is a form characterized by interaction between the said compound in ionic form and a coformer in ionic form (e.g., an acid) via the transfer of one or more protons from the coformer donor to the compound acceptor. The structure of substituted 3-(1-methylpyrrolidin-2-yl)pyridines, optionally substituted 3-(azetidin-2-yl)pyridines, and optionally substituted 3-(azetidin-2-ylmethoxy)pyridines as disclosed herein are such that they comprise two nitrogen atoms that are capable of accepting protons from a coformer and, accordingly, can be present in non-protonated, mono-protonated, and/or di-protonated form in a given sample. Salts of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent can be provided using the types of agents and techniques set forth for nicotine in U.S. Pat. No. 2,033,909 to Cox et al. and Perfetti, Beitrage Tabakforschung Int., 12: 43-54 (1983), which are incorporated herein by reference Suitable salts are generally water soluble. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexanoic acid, trimesic acid, 5-nitroisophthalic acid and zinc chloride monohydrate (forming a hydrated zinc chloride complex salt). In some embodiments, the salt is with an organic acid as described herein below.

In some embodiments, a hydrophilic acid is chosen so as to increase water solubility and/or decrease lipophilicity of the salt. Lipophilicity of a salt of a compound as disclosed herein can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{ionized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified.

When the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt, it is generally preferred that the salt have a relatively low log D, indicative of good water solubility. Without wishing to be bound by theory, it is believed that highly water-soluble salt forms may exhibit a high rate of dissolution, which may be favorable in certain embodiments. Accordingly, in some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent salt has a log D from about −1.0 to about 3 at a pH in a range from about 3 to about 11, such as from about −0.5 to about 2, about −0.3 to about 1, or about −0.1 to about 0.

In some embodiments, the selection of acid used to make a salt is performed on the basis of sensory effects of the salt, such as taste. Surprisingly, according to the present disclosure, it has been found that salts of certain organic acids, such as galactaric acid, offer a better taste sensation relative to salts of acids such as tartaric or phthalic acids.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt with tartaric acid, succinic acid, orotic acid, fumaric acid, pyroglutamic acid, or galactaric acid. In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine is present in the form of a salt with succinic acid or galactaric acid.

The stoichiometry of the salts as described herein can vary. For example, in some embodiments, the stoichiometry can range from about 5:1 to about 1:5 molar equivalents of compound:acid, including any whole or fractional value in between. In some embodiments, the molar ratio of compound to acid is about 2:1, about 1:1, or about 1:2. In some embodiments, the compound is present in the form of a bitartrate salt. Hydrates and other solvates of salts are further contemplated herein.

The salts as described herein can, in some embodiments, exist in various polymorphic and pseudopolymorphic forms. Polymorphism is the ability of a crystalline material to exist in more than one form or crystal structure. Polymorphism can result, e.g., from the existence of different crystal packing structures (packing polymorphism) or from the existence of different conformers of the same molecule (conformational polymorphism). Pseudopolymorphism is the result of hydration or solvation of a material and is also referred to as solvomorphism.

### Organic Acid

In some embodiments, the aerosol generating material comprises an organic acid. In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2-(4-isobutylphenyl)propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-O-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids include, but are not limited to, the list of organic acids in Table 1.

TABLE 1
Non-limiting examples of suitable organic acids
Acid Name

benzoic acid
phenylacetic
p-toluic acid
ethyl benzoic acid
isopropyl benzoic acid
4-phenylbutyric
2-(4-Isobutylphenyl)propanoic acid
2-napthoxyacetic acid
napthylacetic acid
heptanoic acid
octanoic acid
nonanoic acid
decanoic acid
9-deceneoic acid
2-deceneoic acid
10-undecenoic acid
dodecandioic acid
dodecanoic acid
myristic acid
palmitic acid
stearic acid
cyclohexanebutanoic acid
1-heptanesulfonic acid
1-octanesulfonic acid
1-nonanesulfonic acid

The selection of organic acid may depend on certain properties. For example, an organic acid should be one recognized as safe for human consumption, and which has acceptable flavor, odor, volatility, stability, and the like. Determination of appropriate organic acids is within the purview of one of skill in the art. In some embodiments, the acid is benzoic acid. In some embodiments, the acid is levulinic acid. In some embodiments, the acid is lactic acid. In some embodiments, the acid is one or more of benzoic acid, lactic acid, and levulinic acid.

In some embodiments, more than one organic acid may be present. For example, the aerosol modifying agent may comprise two, or three, or four, or more organic acids. Accordingly, reference herein to “an organic acid” contemplates mixtures of two or more organic acids. The relative amounts of the multiple organic acids may vary. For example, an aerosol modifying agent may comprise equal amounts of two, or three, or more organic acids, or may comprise different relative amounts. Without wishing to be bound by theory, it is believed that a combination of different organic acids may provide a concentration of any single organic acid in the aerosol generating material which remains below the threshold which would be found objectionable from a sensory perspective.

The amount of organic acid present in the aerosol generating material, relative to the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine), may vary. In some embodiments, the aerosol generating material comprises from about 0.05, about 0.1, about 1, about 1.5, about 2, or about 5, to about 10, about 15, or about 20 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, calculated as the free base of the 3-(1-methylpyrrolidin-2-yl)pyridine, 3-(azetidin-2-yl)pyridine, or 3-(azetidin-2-ylmethoxy)pyridine.

In some embodiments, the aerosol generating material comprises from about 2 to about 10, or from about 2 to about 5 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, on a free-base basis. In some embodiments, the organic acid is present in a molar ratio with the substituted 3-(1-methylpyrrolidin-2-yl)pyridine, 3-(azetidin-2-yl)pyridine, or 3-(azetidin-2-ylmethoxy)pyridine from about 2, about 3, about 4, or about 5, to about 6, about 7, about 8, about 9, or about 10. In embodiments wherein more than one organic acid it is to be understood that such molar ratios reflect the totality of the organic acids present.

In some embodiments, the aerosol generating material comprises one or more of the organic acids described herein above. The substituted 3-(1-methylpyrrolidin-2-yl)pyridine, 3-(azetidin-2-yl)pyridine, or 3-(azetidin-2-ylmethoxy)pyridine and the one or more organic acids may be present independently, in the form of a salt, or as a cocrystal. In some embodiments, the aerosol generating material comprises one or more of lactic acid, levulinic acid, benzoic acid, succinic acid, galactaric acid, orotic acid, fumaric acid, pyroglutamic acid, and tartaric acid, and at least a portion of said acid is present in the form of a salt with the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a lactic acid salt of a substituted 3-(1-methylpyrrolidin-2-yl)pyridine.

In some embodiments, the aerosol generating material comprises a levulinic acid salt of a substituted 3-(1-methylpyrrolidin-2-yl)pyridine.

In some embodiments, the aerosol generating material comprises a galactaric acid salt of a substituted 3-(1-methylpyrrolidin-2-yl)pyridine.

In some embodiments, the aerosol generating material comprises a tartaric acid salt of a substituted 3-(1-methylpyrrolidin-2-yl)pyridine.

The stoichiometry of such lactate, levulinate, tartrate, and galactarate salts may vary. Accordingly, the molar ratio of lactic, levulinic, tartaric, or galactaric acid to substituted 3-(1-methylpyrrolidin-2-yl)pyridine may be in a range from about 3:1 to about 1:3, including any whole or fractional value in between, such as for example about 2:1, about 1.5:1, about 1:1, about 1:1.5, or about 1:2.

In some embodiments, the aerosol generating material may be substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection).

### Resin Complex

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent may be present in the form of a polymer complex, where the compound is bound to an acidic polymer. The polymer of such a complex can be any polymer (including homopolymers or all types of copolymers) with acidic functionalities, e.g., a polymeric cation exchange resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phosphonic acids, phosphoric acids, iminodiacetic acids, and phenolic groups (e.g., as disclosed in Adams et al., J. Soc. Chem. Ind. 54, IT (1935), which is incorporated herein by reference). Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrin. In some embodiments, the polymer comprises cross-linked moieties. Various acidic ion-exchange resins which are known in the art and are suitable for formation of complexes, include, but are not limited to, polymethacrylic acid resins such as DuPont™ Amberlite™ IRP64, DuPont™ Amberlite™ IRP69, Purolite™ C115HMR, Doshion™ P551, and polyacrylic carbomers, such as Carbopol 974P. See, for example, U.S. Pat. No. 3,901,248 to Lichtneckert et al., which is incorporated herein by reference. In some embodiments, when the substituted 3-(1-methylpyrrolidin-2-yl)pyridine, optionally substituted 3-(azetidin-2-yl)pyridine, or optionally substituted 3-(azetidin-2-ylmethoxy)pyridine is present in the form of a polymer complex, the aerosol generating material further comprises a divalent metal buffer, such as a calcium or magnesium salt (e.g., carbonate, bicarbonate, oxide, acetate, or the like).

### Cocrystal

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent may be present in the form of a co-crystal with at least one other component (“coformer”), both in neutral form. Specifically, as defined in a US FDA industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., a substituted 3-(1-methylpyrrolidin-2-yl)pyridine and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine, optionally substituted 3-(azetidin-2-yl)pyridine, or optionally substituted 3-(azetidin-2-ylmethoxy)pyridine and coformer in a defined stoichiometric ratio. In some embodiments, co-crystals can encompass hydrates, solvates, and clathrates. Co-crystals can comprise the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent in combination with an organic and/or an inorganic coformer.

Examples of suitable coformers include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histadine, L-arginine, L-asparagine, glutamine, L-cysteine, alanine, valine, isoleucine, leucine, morpholine, theronine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid, 3,5-diodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, and 2,3,4,5-tetrafluoro-6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (p-resorcylic acid), 2,5-dihydroxybenzoic acid (gentisic acid/hydroquinonecarboxylic acid), 2,6-dihydroxybenzoic acid (γ-resorcylic acid), 3,4-dihydroxybenzoic acid (protocatechuic acid), 3,5-dihydroxybenzoic acid (α-resorcylic acid), 4-hydroxy-3-methoxybenzoic acid (vanillic acid), 6-methyl-2,4-dihydroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro-2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid). Further contemplated are various combinations of any of the foregoing acids.

In some embodiments, the coformer is L-malic acid, succinic acid, or a combination thereof. In some embodiments, the coformer is 1,1,6,6-tetraphenyl-2,4-hexidiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt co-crystal. A “salt co-crystal” is a type of hybrid structure with both salt and co-crystal characteristics. Typically, a substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent within a salt co-crystal is associated with at least two coformers (which may be the same or different), wherein one coformer is in ionic form (e.g., an acid) and transfers a proton to the substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent, and wherein a second coformer does not transfer a proton to the substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent. Suitable acids and coformers are generally those described herein above with respect to salts and co-crystals.

The stoichiometry of the co-crystals and salt co-crystals described herein can vary. For example, in certain embodiments, where two components are present, the stoichiometry can range in certain embodiments from about 5:1 to about 1:5 compound:coformer. Where more than one coformer is used to form a co-crystal or salt co-crystal, the ratios of the coformers with respect to both the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent and to one another can also vary.

The co-crystals and salt co-crystals described herein can, in some embodiments, exist in various polymorphic and pseudopolymorphic forms, as well as solvates and hydrates.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt-co-crystal. In some embodiments, the salt-co-crystal is a bis-orotic acid salt-co-crystal. In some embodiments, the bis-orotic acid salt-co-crystal is a hemi-hydrate.

### Ion Pairing

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (e.g., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the aerosol generating material as disclosed herein, and/or upon oral use of said aerosol generating material (e.g., upon contact with saliva), the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed aerosol generating material, both before and during use by the consumer, may vary based on, for example, pH, the nature of the acid, the concentration of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent, the concentration of the acid or conjugate base of the acid present in the aerosol generating material, the moisture content of the aerosol generating material, the ionic strength of the aerosol generating material, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent between octanol and water, or by performing membrane permeation studies of aqueous solutions of, for example, the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent through the oral mucosa. However, as described above, in some embodiments, the properties of the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent are such that no ion pairing is required, and accordingly, the aerosol generating material is substantially or completely free of any ion pairing. By “substantially free” it is meant that no measurable degree of ion pairing is present.

In embodiments where ion pairing is desired, the aerosol generating material comprises an organic acid, an alkali metal salt of an organic acid, or both. In such embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific aerosol generating material agent as opposed to merely being inherently present as a component of another aerosol generating material agent (e.g., the small amount of organic acid which may inherently be present in a composition ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the aerosol generating material of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the aerosol generating material may be characterized as substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection). This is not to be interpreted as meaning that the aerosol generating material is substantially or completely free of substituted 3-(1-methylpyrrolidin-2-yl)pyridine salts or substituted 3-(1-methylpyrrolidin-2-yl)pyridine co-crystals unless explicitly recited.

The amount of organic acid or alkali metal salt thereof present in the aerosol generating material, relative to the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent, may vary. Generally, as the concentration of the organic acid (or the conjugate base thereof) increases, the percent of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent that is ion paired with the organic acid increases. This typically increases the partitioning of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent in the form of an ion pair, into octanol versus water as measured by the log P (the log₁₀ of the partitioning coefficient). In some embodiments, the aerosol generating material comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent, calculated as the free base of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent.

In some embodiments, the aerosol generating material comprises from about 2 to about 10, or from about 2 to about 5 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent on a free-base basis. In some embodiments, the organic acid, the alkali metal salt thereof, or the combination thereof, is present in a molar ratio with the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the aerosol generating material comprises benzoic acid and sodium benzoate wherein a total amount of benzoate (i.e., benzoic acid and benzoate) is in a molar ratio in a range from about 3 to about 5 relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent. In some embodiments, the molar ratio of the total amount of benzoate to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is about 3.2 or about 4.8.

In some embodiments, the organic acid inclusion is sufficient to provide a pH of from about 4.0 to about 9.0, such as from about 4.5 to about 7.0, or from about 5.5 to about 7.0, from about 4.0 to about 5.5, or from about 7.0 to about 9.0. Reference herein to “a pH” means the pH of an aqueous solution of the aerosol generating material prepared by dissolving or suspending 5 grams of aerosol generating material in 95 grams of water and measuring the pH of the resulting solution with a calibrated pH meter.

In some embodiments, the organic acid inclusion is sufficient to provide a pH of from about 4.5 to about 6.5, for example, from about 4.5, about 5.0, or about 5.5, to about 6.0, or about 6.5. In some embodiments, the desired pH is from about 4.5 to about 6.5, and the organic acid is provided in a quantity sufficient to provide such a pH. In some embodiments, the organic acid is provided in a quantity sufficient to provide a pH of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the aerosol generating material to the desired value. In some embodiments, a buffer (e.g., a buffer as described herein below) is added to the aerosol generating material to the desired value, and/or to maintain the pH of the aerosol generating material at the desired value.

In some embodiments, the aerosol generating material further comprises a solubility enhancer to increase the solubility of one or more of the organic acid or salt thereof. Suitable solubility enhancers include, but are not limited to, humectants as described herein, such as glycerol or propylene glycol.

### Other Active Agents

In some embodiments, the aerosol generating material comprises an active agent in addition to the active agents described herein above. These additional active agents, include, but are not limited to, botanical extracts, cannabinoids, terpenes, tobacco extracts, and combinations thereof. The additional active agent may be a physiologically active material, which is a material intended to achieve or enhance a physiological response. The active agent may for example be selected from nutraceuticals, nootropics, and psychoactives. The active agent may be naturally occurring or synthetically obtained. The active agent may comprise for example nicotine, caffeine, taurine, theanine, vitamins such as B6 or B12 or C, melatonin, cannabinoids, or constituents, derivatives, or combinations thereof. The active agent may comprise one or more constituents, derivatives or extracts of cannabis or another botanical material or extract thereof (other than tobacco).

### Non-Tobacco Botanical Material

In some embodiments, the other active agent comprises a non-tobacco botanical material. As used herein, the term “botanical” includes any material derived from plants including, but not limited to, extracts, leaves, bark, fibers, stems, roots, seeds, flowers, fruits, pollen, husk, shells or the like. A “non-tobacco botanical material” is a botanical material other than tobacco. The use of a non-tobacco botanical material may enhance the sensorial properties of an aerosol produced by the aerosol generating material. The material may be in the form of liquid, gas, solid, powder, dust, crushed particles, granules, pellets, shreds, strips, sheets, or the like. In some embodiments, the botanical material is in the form of a solid. In some embodiments, the botanical material comprises leaf material. The botanical material may be a particulate or granular material. In some embodiments, the non-tobacco botanical material is a powder. The non-tobacco botanical material may be formed by grinding solid non-tobacco botanical material to produce a ground non-tobacco botanical material. Alternatively or in addition, the non-tobacco botanical material may comprise strips, strands or fibers of non-tobacco botanical material. For example, the non-tobacco botanical material may comprise particles, granules, fibers, strips and/or strands of non-tobacco botanical material. In some embodiments, the non-tobacco botanical material consists of particles or granules of non-tobacco botanical material. In some embodiments, the non-tobacco botanical material is in a particulate or ground form.

Example non-tobacco botanical material include, but are not limited to, eucalyptus, star anise, hemp, cocoa, cannabis, fennel, lemongrass, peppermint, spearmint, Aspalathus linearis (rooibos; red or green), chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens.

In some embodiments, the other active agent comprises or is derived from one or more botanicals or constituents, derivatives or extracts thereof, and the botanical is tobacco. In some embodiments, the aerosol generating material is substantially free from tobacco. By “substantially free from” it is meant that the material comprises less than 1 wt %, such as less than 0.5 wt % tobacco. In some embodiments, the aerosol generating material is free from tobacco. In some embodiments, the aerosol generating material does not comprise tobacco fibers. In particular embodiments, the aerosol generating material does not comprise fibrous material.

In some embodiments, the other active agent comprises or is derived from one or more botanicals or constituents, derivatives or extracts thereof. In some embodiments, the other active agent comprises or is derived from one or more botanicals or constituents, derivatives or extracts thereof, and the botanical is selected from eucalyptus, star anise, cocoa and hemp.

### Nicotine

In some embodiments, the aerosol generating material comprises nicotine as an active agent. In some embodiments, the aerosol generating material comprises no tobacco material but does comprise nicotine. In some such cases, the aerosol generating material may comprise from about 1 wt %, 2 wt %, 3 wt % or 4 wt % to about 20 wt %, 18 wt %, 15 wt % or 12 wt % (calculated on a dry weight basis) of nicotine. For example, the aerosol generating material may comprise 1-20 wt %, 2-18 wt % or 3-12 wt % of nicotine. In some embodiments, the aerosol generating material has a nicotine content of from about 1.5 wt % to about 7 wt % (DWB).

In some embodiments, the aerosol generating material comprises from about 1 wt %, about 1.5 wt % or about 2 wt % to about 6 wt %, about 5 wt %, about 4 wt % or about 3 wt % of nicotine (DWB).

In some embodiments, the aerosol generating material of the disclosure can be completely free or substantially free of nicotine (3-(1-methylpyrrolidin-2-yl)pyridine). For example, some embodiments can have less than 0.01% by weight of nicotine, or less than 0.001% by weight of nicotine, or less than 0.0001%, or even 0% by weight of nicotine, calculated as the free base and based on the total weight of the aerosol generating material. In some embodiments the aerosol generating material is completely free of (R)-, (S)-, and (R/S)-3-(1-methylpyrrolidin-2-yl)pyridine (e.g., having 0% by weight of nicotine, including racemic nicotine and nicotine enantiomers, calculated as the free base and based on the total weight of the aerosol generating material).

### Cannabinoids

In some embodiments, the other active agent comprises one or more cannabinoids. As used herein, the term “cannabinoid” refers to a class of diverse natural or synthetic chemical compounds that acts on cannabinoid receptors (e.g., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

In certain embodiments, the cannabinoid is selected from tetrahydrocannabinol (THC), the primary psychoactive compound in cannabis, and cannabidiol (CBD), another major constituent of the plant, but which is devoid of psychoactivity. All of the above compounds can be used in the form of an isolate from plant material or synthetically derived. Certain cannabinoids, including but not limited to CBD and THC, may exist in more than one isomeric form, for example Δ8- and Δ9-THC. Such isomeric forms may be naturally occurring or may be synthetic. For avoidance of doubt, reference within the present disclosure to a “cannabinoid” is intended to be inclusive of any and all isomeric forms thereof.

In some embodiments, the cannabinoid comprises at least tetrahydrocannabinol (THC). In some embodiments, the cannabinoid is tetrahydrocannabinol (THC). In some embodiments, the THC is Δ8-THC. In some embodiments, the THC is Δ9-THC.

In some embodiments, the cannabinoid comprises at least cannabidiol (CBD). In some embodiments, the cannabinoid is cannabidiol (CBD). In some embodiments, the CBD is synthetic CBD. In some embodiments, the CBD is Δ8-CBD. In some embodiments, the CBD is Δ9-CBD.

In some embodiments, the cannabinoid (e.g., CBD) is added to the aerosol generating material in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the aerosol generating material is no greater than about 1% by weight of the aerosol generating material, such as no greater than about 0.5% by weight of the aerosol generating material such as no greater than about 0.1% by weight of the aerosol generating aerosol generating material, such as no greater than about 0.01% by weight of the aerosol generating material.

The choice of cannabinoid and the particular percentages thereof which may be present within the aerosol generating material will vary depending upon the desired characteristics of the material.

In some embodiments, the cannabinoid (such as CBD) is present in the aerosol generating material in a concentration of at least about 0.001% by weight of the aerosol generating material, such as in a range from about 0.001% to about 2% by weight of the aerosol generating material. In some embodiments, the cannabinoid (such as CBD) is present in the aerosol generating material in a concentration of from about 0.1% to about 1.5% by weight, based on the total weight of the aerosol generating material. In some embodiments, the cannabinoid (such as CBD) is present in a concentration from about 0.4% to about 1.5% by weight, based on the total weight of the aerosol generating material.

Alternatively, or in addition to a cannabinoid, the other active agent may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

### Terpenes

Other active agents suitable for use in the aerosol generating material can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active agent comprises more than one terpene. For example, the active agent may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

Terpenes and/or cannabinoids may be present as an active agent, as an aerosol former, or as a flavorant. The amount of terpene and/or cannabinoid present may vary accordingly based on their intended purpose.

In some embodiments, the active agent comprises caffeine, melatonin, an amino acid, a vitamin, or combinations thereof. In some embodiments, the active agent comprises taurine, theanine, vitamin B6, B12, or C, melatonin, cannabinoids, or constituents, derivatives, or combinations thereof.

### Flavoring Agent

In some embodiments, the aerosol generating material as described herein comprises a flavoring agent. As used herein, a “flavoring agent” or “flavorant” is any flavorful or aromatic substance capable of altering the sensory characteristics associated with the aerosol generating material or the vapor produced therefrom. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may be imitation, synthetic or natural ingredients or blends thereof. Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the aerosol generating material comprises a sensate which provides to the user of such aerosol generating material a cooling effect. Suitable cooling agents include, but are not limited to, menthane, menthone, menthone ketals, menthone glycerol ketals, substituted p-menthanes, acyclic carboxamides, monomenthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA4693), WS-116 (FEMA4603), N-ethyl-2,2-diisopropylbutanamide, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, menthyl glutarate, N,2,3-trimethyl-2-(1-methylethyl)-butanamide, N-ethyl-trans-2-cis-6-nonadienamide, N,N-dimethyl menthyl succinamide, N-(2-hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)-N-(4-(carbamoylmethyl)phenyl)-menthylcarboxamide, 2-[2-(p-menthan-3-yloxy)ethoxy]ethanol, (1R,2R,4R)-1-(2-Hydroxy-4-methylcyclohexyl)ethenone, 2-(p-tolyloxy)-N-(1H-pyrazol-5-yl)-N-((thiophen-2-yl)methyl)acetamide, menthol methyl ether, menthyl pyrrolidone carboxylate, 2,5-dimethyl-4-(1-pyrrolidinyl)-3(2H)-furanone, cyclic a-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one). Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which are incorporated in their entirety by reference hereto. In some embodiments, the cooling agent comprises menthol, eucalyptus, mint, menthol, menthyl esters, eucolyptol, WS-3, WS-23, WS-5, (1R,2S,5R)-N-(4-(cyanomethyl)phenyl)menthylcarboxamide (Evercool™ 180), (1R,2S,5R)-N-(2-(pyridin-2-yl)ethyl)menthylcarboxamide (Evercool™ 190), or a combination thereof.

In some embodiments, the aerosol generating material does not comprise a flavoring agent, and comprises only a cooling agent to provide the desired user experience. In some embodiments, the cooling agent is WS-3.

In some embodiments, the aerosol generating material comprises a modulator or sensate which provides to the user of such aerosol generating material a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form.

The amount of flavoring agent utilized in the aerosol generating material can vary, but is typically up to about 60% by weight. For example, aerosol generating material may comprise up to about 60 wt %, about 50 wt %, about 40 wt %, about 30 wt %, about 20 wt %, about 10 wt % or about 5 wt % of a flavoring agent. In some embodiments, the aerosol generating material may comprise at least about 0.5 wt %, about 1 wt %, about 2 wt %, about 5 wt %, about 10 wt %, about 20 wt % or about 30 wt % of flavoring agent (calculated on a dry weight basis). For example, the aerosol generating material may comprise from about 10 to about 60 wt %, from about 20 to about 50 wt % or from about 30 to about 40 wt % of flavoring agent.

In some embodiments, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavoring agent (if present) comprises, consists essentially of, or consists of, menthol. In some embodiments, the aerosol generating material does not comprise an added flavor.

### Colorant

In some embodiments, the aerosol generating material comprise a colorant. The addition of a colorant may alter the visual appearance of the aerosol generating material. The presence of colorant in the aerosol generating material may enhance the visual appearance of the aerosol generating material. By adding a colorant to the aerosol generating material, the aerosol generating material may be color-matched to other components of an article comprising the aerosol generating material.

A variety of colorants may be used depending on the desired color of the aerosol generating material. The color of aerosol generating material may be, for example, white, green, red, purple, blue, brown or black. Other colors are also envisaged. Natural or synthetic colorants, such as natural or synthetic dyes, food-grade colorants and pharmaceutical-grade colorants may be used. In certain embodiments, the colorant is caramel, which may confer the aerosol generating material with a brown appearance. In such embodiments, the color of the aerosol generating material may be similar to the color of other components (such as tobacco material) in an aerosol generating aerosol generating material comprising the aerosol generating material. In some embodiments, the addition of a colorant to the aerosol generating material renders it visually indistinguishable from other components in the aerosol generating material.

The colorant may be incorporated during the formation of the aerosol generating material (e.g. when forming a slurry comprising the materials that form the aerosol generating material) or it may be applied to the aerosol generating material after its formation (e.g. by spraying it onto the aerosol generating material).

### Water

The aerosol generating material may have any suitable water content, such as from 1 wt % to 15 wt %. Suitably, the water content of the aerosol generating material may be from about 5 wt %, 7 wt % or 9 wt % to about 15 wt %, 13 wt % or 11 wt % (wet weight basis) (WWB). The water content of the aerosol generating material may, for example, be determined by Karl-Fischer-titration or Gas Chromatography with Thermal Conductivity Detector (GC-TCD).

### Functional Materials

The one or more other functional materials may comprise one or more of pH regulators, preservatives, stabilizers, and/or antioxidants.

### Form of the Aerosol Generating Material

The form of the aerosol generating material may vary. For example, the aerosol generating material may be a homogeneous solid. The aerosol generating material may be an “amorphous solid”. In some embodiments, the amorphous solid is a “monolithic solid”. The aerosol generating material may be non-fibrous or fibrous. In some embodiments, the aerosol generating material may be a dried gel. The aerosol generating material may be a solid material that may retain some fluid, such as liquid, within it. In some embodiments the retained fluid may be water (such as water absorbed from the surroundings of the aerosol generating material) or the retained fluid may be solvent (such as when the aerosol generating material is formed from a slurry). In some embodiments, the solvent may be water.

The consumable of the disclosure comprises one or more discrete portions of the aerosol generating material. In some embodiments, the consumable may comprise from about 1 to about 15 discrete portions of aerosol generating material. In some embodiments, the consumable may comprise from about 1 to about 10 discrete portions, such as from about 2 to about 8 discrete portions or from about 2 to about 6 discrete portions. In some embodiments, the consumable comprises at least two discrete portions of aerosol generating material.

In some embodiments, the discrete portions of aerosol generating material are located on a carrier. The discrete portions of aerosol generating material may be present on or in a carrier support (or carrier component). The carrier may function as a support on which the aerosol generating material is formed, thereby easing manufacture. The carrier may also provide rigidity to the aerosol generating material, easing handling.

The carrier may be any suitable material which can be used to support an aerosol generating material. In some cases, the carrier may be formed from materials selected from metal foil, paper, carbon paper, greaseproof paper, ceramic, carbon allotropes such as graphite and graphene, plastic, cardboard, wood or combinations thereof. In some cases, the carrier may comprise or consist of a tobacco material, such as a sheet of reconstituted tobacco. In some cases, the carrier may be formed from materials selected from metal foil, paper, cardboard, wood or combinations thereof. In some cases, the carrier comprises paper. In some cases, the carrier itself may be a laminate structure comprising layers of materials selected from the preceding lists. In some cases, the carrier may also function as a flavor support. For example, the carrier may be impregnated with a flavorant or with tobacco extract.

In some cases, the carrier may be magnetic. This functionality may be used to fasten the carrier to the assembly in use, or may be used to generate particular aerosol generating material shapes. In some cases, the consumable may comprise one or more magnets which can be used to fasten the consumable to an induction heater in use.

In some cases, the carrier may be substantially or wholly impermeable to gas and/or aerosol. This prevents aerosol or gas passage through the carrier layer, thereby controlling the flow and ensuring it is delivered to the user. This can also be used to prevent condensation or other deposition of the gas/aerosol in use on, for example, the surface of a heater provided in an aerosol generating assembly. Thus, consumption efficiency and hygiene can be improved in some cases.

In some cases, the surface of the carrier that abuts the aerosol generating material may be porous. For example, in one case, the carrier comprises paper. A porous carrier such as paper is particularly suitable for the present invention; the porous (e.g. paper) layer abuts the aerosol generating layer and forms a strong bond. The aerosol generating material is formed by drying a gel and, without being limited by theory, it is thought that the slurry from which the gel is formed partially impregnates the porous carrier (e.g. paper) so that when the gel sets and forms cross-links, the carrier is partially bound into the gel. This provides a strong binding between the gel and the carrier (and between the dried gel and the carrier).

Additionally, surface roughness may contribute to the strength of bond between the aerosol generating material and the carrier. The paper roughness (for the surface abutting the carrier) may suitably be in the range of 50-1000 Bekk seconds, suitably 50-150 Bekk seconds, suitably 100 Bekk seconds (measured over an air pressure interval of 50.66-48.00 kPa). (A Bekk smoothness tester is an instrument used to determine the smoothness of a paper surface, in which air at a specified pressure is leaked between a smooth glass surface and a paper sample, and the time (in seconds) for a fixed volume of air to seep between these surfaces is the “Bekk smoothness”.)

Conversely, the surface of the carrier facing away from the aerosol generating material may be arranged in contact with the heater, and a smoother surface may provide more efficient heat transfer. Thus, in some cases, the carrier is disposed so as to have a rougher side abutting the aerosol generating material and a smoother side facing away from the aerosol generating material.

In one particular case, the carrier may be a paper-backed foil; the paper layer abuts the aerosol generating material layer and the properties discussed in the previous paragraphs are afforded by this abutment. The foil backing is substantially impermeable, providing control of the aerosol flow path. A metal foil backing may also serve to conduct heat to the aerosol generating material. In another case, the foil layer of the paper-backed foil abuts the aerosol generating material. The foil is substantially impermeable, thereby preventing water provided in the aerosol generating material to be absorbed into the paper which could weaken its structural integrity.

In some cases, the carrier is formed from or comprises metal foil, such as aluminum foil. A metallic carrier may allow for better conduction of thermal energy to the aerosol generating material. Additionally, or alternatively, a metal foil may function as a susceptor in an induction heating system. A susceptor is a material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically-conductive and magnetic, so that the susceptor is heatable by both heating mechanisms.

In particular embodiments, the carrier comprises a metal foil layer and a carrier layer, such as cardboard. In these embodiments, the metal foil layer may have a thickness of less than 20 μm, such as from about 1 μm to about 10 μm, suitably about 5 μm. In some cases, the carrier may have a thickness of between about 0.017 mm and about 2.0 mm, suitably from about 0.02 mm, 0.05 mm or 0.1 mm to about 1.5 mm, 1.0 mm, or 0.5 mm.

In some embodiments from about 1 to about 15 discrete portions of aerosol generating material are disposed on a surface of the carrier, such as from about 1 to about 10 discrete portions, from about 2 to about 8 discrete portions, or from about 2 to about 6 discrete portions. In some embodiments, the discrete portions of aerosol generating material are disposed in a 1 to 3 by N array, wherein N depends on the number of discrete portions disposed on the carrier.

In embodiments where the discrete portions of aerosol generating material are deposited on a carrier, the discrete portions of aerosol generating material are separated from one another such that each of the discrete portions may be energized (e.g. heated) individually or selectively to produce an aerosol.

In some embodiments, the discrete portions of aerosol generating material are substantially round, cylindrical or hemispherical. The discrete portions may take any other shape such as square or rectangle. In some embodiments the discrete portions of aerosol generating material are in the form of dots, stripes or lines.

In some embodiments, each of the discrete portions of aerosol generating material may have a mass of no greater than 20 mg, such that the amount of material to be aerosolized by a given aerosol generating component at any one time is relatively low. For example, the mass per portion may be equal to or lower than 20 mg, or equal to or lower than 10 mg, or equal to or lower than 5 mg.

Each of the discrete portions of aerosol generating material may have a mass of from about 0.1 to about 4 mg. In some embodiments, each of the discrete portions of aerosol generating material has a mass of from about 0.5 to about 3 mg, from about 0.5 to about 2 mg or from about 0.5 to about 1 mg.

In some embodiments, the one or more discrete portions of aerosol generating material have the same aerosol generating material. In some embodiments, at least two discrete portions of aerosol generating material are different from one another. The aerosol generating materials may vary in, for example, active agent, aerosol former, binder, filler, and/or in the concentration of any thereof. When the discrete portions of aerosol generating material are different from one another, this may allow a user to customize the aerosol that is received per inhalation or per inhalation session.

In some embodiments, the weight of each discrete portion of aerosol generating material within the consumable varies by no more than about 10%, such as no more than about 8%, about 7%, about 5% or about 3%.

In some embodiments, each discrete portion of aerosol generating material within the consumable has the same or substantially the same weight. In some embodiments, the total amount of active agent in any given discrete portion is within about 10% of the total amount of active agent in any of the other discrete portions. That is, the amount of active agent (e.g., substituted 3-(1-methylpyrrolidin-2-yl)pyridine) in any given discrete portion is from about 90% to about 110% of the of any of the total amount of active agent in any other discrete portion.

In some embodiments, the total amount of active agent in any given discrete portion is within about 5%, about 4%, about 3% or about 2% of the total amount of active agent in any of the other discrete portions.

In some embodiments, the consumable comprises at least two discrete portions of aerosol generating material, and the weight of any given discrete portion is within about 10% of the weight of any of the other discrete portions. That is, the weight of any given discrete portion is from about 90% to about 110% of the weight of any of the other discrete portions.

In some embodiments, the total weight of any given discrete portion is within about 5%, about 4%, about 3% or about 2% of the total weight of any of the other discrete portions.

In some embodiments, the consumable comprises at least two discrete portions of aerosol generating material, a first discrete portion of aerosol generating material has a weight of from about 1 to about 20 mg, and the weight of each of the other discrete portions is between about 90% and about 100% of the weight of the first discrete portion.

In some embodiments, at least 70 wt %, 80 wt %, 90 wt %, 95 wt % or 100 wt % of the one or more of the active agent is aerosolized during use, for example when the aerosol generating material is heated to a temperature of less than or equal to 350° C., such as from about 220° C. to about 280° C. or from about 250° C. to about 280° C., for a period of at least 1 second, such as from about 1 second to about 5 seconds or from about 1 second to about 3 seconds. In some embodiments, at least 70 wt %, 80 wt %, 90 wt %, 95 wt % or 100 wt % of the active agent contained in a single discrete portion of the aerosol generating material is aerosolized during a single puff. A single puff may be from about 1 second to about 5 seconds at a temperature of less than or equal to 350° C., such as from about 220° C. to about 280° C. or from about 250° C. to about 280° C. For example, a single puff may be 3 seconds at a temperature of less than or equal to 350° C., such as from about 220° C. to about 280° C. or from about 250° C. to about 280° C.

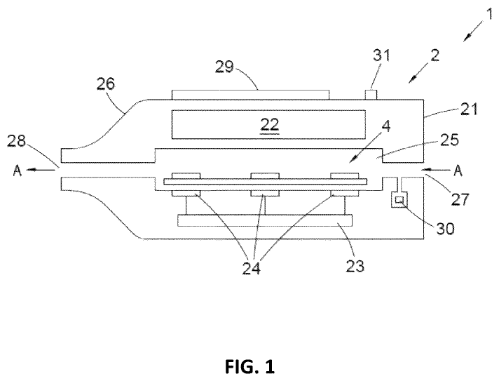

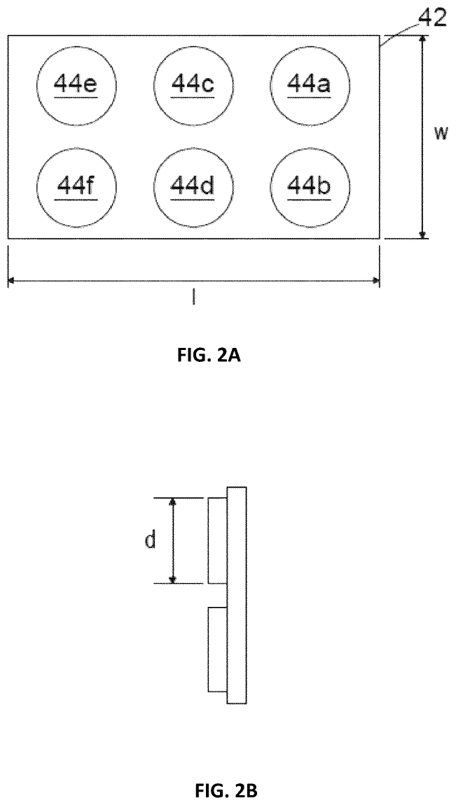

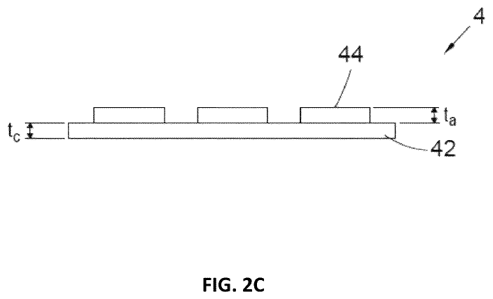

An example consumable (also called an aerosol generating article) 4 (as shown in FIG. 1 discussed below) comprising a carrier 42 is shown in FIGS. 2A, 2B, and 2C. FIG. 2A is a top-down view of the article 4, FIG. 2B is an end-on view along the longitudinal (length) axis of the article 4, and FIG. 2C is a side-on view along the width axis of the article 4.

The discrete portions of aerosol generating material may have a diameter d and a thickness tₐ as shown in FIGS. 2A, 2B, and 2C. The thickness of the discrete portions of the aerosol generating material may be any suitable thickness. For example, the thickness tₐ may be in the range of 50 pm to 1.5 mm. In some embodiments, the thickness tₐ is from about 50 pm to about 200 pm, or about 50 pm to about 100 pm, or about 60 pm to about 90 pm, suitably about 77 pm. In other embodiments, the thickness ta may be greater than 200 pm, e.g., from about 50 pm to about 400 pm, or to about 1 mm, or to about 1.5 mm.

In some cases, the carrier 42 is broadly cuboidal in shape and has a length L, a width W and a thickness t_{c} as shown in FIGS. 2A, 2B, and 2C. The length may be from about 10 mm to about 150 mm, such as from about 20 mm to about 100 mm or from about 30 mm to about 80 mm. The width may be from about 4 mm to about 40 mm, such as from about 5 mm to about 30 mm or from about 7 mm to about 25 mm.

By way of a concrete example, the length of the carrier component 42 may be 30 to 80 mm, the width may be 7 to 25 mm, and the thickness may be between 0.2 to 1 mm. However, it should be appreciated that the above are exemplary dimensions of the carrier component 42, and in other embodiments the carrier component 42 may have different dimensions as appropriate. In some embodiments, the carrier component 42 may comprise one or more protrusions extending in the length and/or width directions of the carrier component 42 to help facilitate handling of the article 4 by the user.

Suitably, the thickness of the carrier layer may be in the range of about 10 μm, 15 μm, 17 μm, 20 μm, 23 μm, 25 μm, 50 μm, 75 μm or 0.1 mm to about 2.5 mm, 2.0 mm, 1.5 mm, 1.0 mm or 0.5 mm. The carrier may comprise more than one layer, and the thickness described herein refers to the aggregate thickness of those layers.

### Method of Making Aerosol Generating Material

In another aspect is provided a method of forming one or more discrete portions of aerosol generating material as disclosed herein. The method generally comprises:
(a) providing a slurry comprising:
   (i) an aerosol former;
   (ii) binder;
   (iii) active agent(s); and
   (iv) solvent;
(b) forming one or more discrete portions of the slurry; and
(c) drying the one or more discrete portions of the slurry to form one or more discrete portions of the aerosol generating material.

This method may be used to form a consumable as disclosed herein. Accordingly, in some embodiments, the method further comprises in (b) forming one or more discrete portions of the slurry on a carrier. In this embodiment, the overall consumable will comprise a carrier, as described herein.

In some embodiments, the slurry comprises the active agent (e.g., a substituted 3-(1-methylpyrrolidin-2-yl)pyridine, 3-(azetidin-2-yl)pyridine, or 3-(azetidin-2-ylmethoxy)pyridine as described herein). In some embodiments, the slurry does not include the active agent, and the active agent is added as a “top dressing” to the discrete portions of the aerosol generating material before or after drying.

In some embodiments, (b) comprises casting the slurry, for example on a carrier.

In some embodiments, drying (c) may, in some cases, remove from about 50 wt %, 60 wt %, 70 wt %, 80 wt % or 90 wt % to about 80 wt %, 90 wt % or 95 wt % (WWB) of water in the slurry.

In some embodiments, (c) may, in some cases, may reduce the material thickness by at least 80%, suitably 85% or 87%. For instance, the slurry may be cast at a thickness of 2 mm, and the resulting dried aerosol generating material may have a thickness of 0.2 mm.

In some embodiments, the slurry may be heated in (c) to remove at least about 60 wt %, 70 wt %, 80 wt %, 85 wt % or 90 wt % of the solvent.

In some embodiments, the slurry solvent may consist essentially of or consist of water. In some cases, the slurry may comprise from about 50 wt %, 60 wt %, 70 wt %, 80 wt % or 90 wt % of solvent (WWB). In embodiments where the solvent consists of water, the dry weight content of the slurry may match the dry weight content of the aerosol generating material.

In some embodiments, the active agent (e.g., a substituted 3-(1-methylpyrrolidin-2-yl)pyridine as described herein) is added to the discrete portions of the aerosol generating material. For example, in some embodiments, at least a portion or even all of the active agent may be applied during or after drying (e.g., by spraying it on or wiping it onto the discrete portions of the aerosol generating material). The active agent is generally applied as a solution in, for example, an aerosol former such as glycerol, propylene glycol, or a mixture thereof. Without wishing to be bound by theory, it is believed that adding the active agent solely after formation of the aerosol generating material, either during or after drying, may reduce the potential for contamination and/or increase the homogeneity of distribution and the amount of the active agent in each discrete portion of aerosol generating material.

### Aerosol Provisional System

In another aspect is provided a non-combustible aerosol provision system comprising a consumable as described herein and a non-combustible aerosol provision device. A non-combustible aerosol provision system may also be referred to as an aerosol generating assembly. A non-combustible aerosol provision device may be referred to as an aerosol generating apparatus.

A non-combustible aerosol provision system is one where a constituent aerosolizable material of the aerosol provision system (or component thereof) is not combusted or burned in order to facilitate delivery of an aerosol to a user. Furthermore, and as is common in the technical field, the terms “vapor” and “aerosol”, and related terms such as “vaporize”, “volatilize” and “aerosolize”, may generally be used interchangeably.

In some embodiments, the non-combustible aerosol provision system is a powered non-combustible aerosol provision system.

In some embodiments, the non-combustible aerosol provision system is an aerosol generating material heating system, also known as a heat-not-burn system. An example of such a system is a tobacco heating system.

In some embodiments, the non-combustible aerosol provision device is a heat-not-burn device.

Non-combustible aerosol provision systems often, though not always, comprise a modular assembly including both a reusable aerosol provision device and a replaceable article (also called a consumable). In some embodiments, the non-combustible aerosol provision device may comprise a power source and a controller (or control circuitry). The power source may, for example, be an electric power source, such as a battery or rechargeable battery, or an exothermic power source. In some embodiments, the exothermic power source comprises a carbon substrate which may be energized so as to distribute power in the form of heat to an aerosol generating material or to a heat transfer material in proximity to the exothermic power source. In some embodiments, the non-combustible aerosol provision device may also comprise an aerosol generating component, also referred to as an “aerosol generator.”

An aerosol generator is an apparatus configured to cause aerosol to be generated from the aerosol generating material. In some embodiments, the aerosol generator is configured to cause an aerosol to be generated from the aerosol generating material without heating. For example, the aerosol generator may be configured to subject the aerosol generating material to one or more of vibration, increased pressure, or electrostatic energy.

In some embodiments, the aerosol generator is a heater configured to subject the aerosol generating material to heat energy, so as to release one or more volatiles from the aerosol generating material to form an aerosol.

The heater is configured to heat not burn the consumable, and thus the aerosol generating material. The heater may be, in some cases, a thin film, electrically resistive heater. In other cases, the heater may comprise an induction heater or the like. The heater may be a combustible heat source or a chemical heat source which undergoes an exothermic reaction to produce heat in use. The aerosol generating assembly may comprise a plurality of heaters. The heater(s) may be powered by a battery.

The provision system may additionally comprise a cooling element and/or a filter. The cooling element, if present, may act or function to cool gaseous or aerosol components. In some cases, it may act to cool gaseous components such that they condense to form an aerosol. It may also act to space the very hot parts of the non-combustible aerosol provision device from the user. The filter, if present, may comprise any suitable filter known in the art such as a cellulose acetate plug.

FIG. 1 is a cross-sectional view through a schematic representation of a noncombustible aerosol provision system 1 in accordance with non-limiting embodiments of the disclosure. The aerosol provision system 1 comprises two main components, namely an aerosol provision device 2 and a consumable (also called an aerosol generating article) 4. The aerosol provision device 2 comprises an outer housing 21, a power source 22, control circuitry 23, a plurality of aerosol generating components 24, a receptacle 25, a mouthpiece end 26, an air inlet 27, an air outlet 28, a touch-sensitive panel 29, an inhalation sensor 30, and an end of use indicator 31.

The outer housing 21 may be formed from any suitable material, for example a plastic material. The outer housing 21 is arranged such that the power source 22, control circuitry 23, aerosol generating components 24, receptacle 25 and inhalation sensor 30 are located within the outer housing 21. The outer housing 21 also defines the air inlet 27 and air outlet 28, described in more detail below. The touch sensitive panel 29 and end of use indicator are located on the exterior of the outer housing 21.

The outer housing 21 further includes a mouthpiece end 26. The outer housing 21 and mouthpiece end 26 are formed as a single component (that is, the mouthpiece end 26 forms a part of the outer housing 21). The mouthpiece end 26 is defined as a region of the outer housing 21 which includes the air outlet 28 and is shaped in such a way that a user may comfortably place their lips around the mouthpiece end 26 to engage with air outlet 28. In FIG. 1, the thickness of the outer housing 21 decreases towards the air outlet 28 to provide a relatively thinner portion of the device 2 which may be more easily accommodated by the lips of a user. In other embodiments, however, the mouthpiece end 26 may be a removable component that is separate from but able to be coupled to the outer housing 21, and may be removed for cleaning and/or replacement with another mouthpiece end 26.

The power source 22 is configured to provide operating power to the aerosol provision device 2. The power source 22 may be any suitable power source, such as a battery. For example, the power source 22 may comprise a rechargeable battery, such as a lithium-ion battery. The power source 22 may be removable or form an integrated part of the aerosol provision device 2. In some embodiments, the power source 22 may be recharged through connection of the device 2 to an external power supply (such as mains power) through an associated connection port, such as a USB port (not shown) or via a suitable wireless receiver (not shown).

The control circuitry 23 is suitably configured/programmed to control the operation of the aerosol provision device to provide certain operating functions of aerosol provision device 2. The control circuitry 23 may be considered to logically comprise various sub-units/circuitry elements associated with different aspects of the aerosol provision devices' operation. For example, the control circuitry 23 may comprise a logical sub-unit for controlling the recharging of the power source 22. Additionally, the control circuitry 23 may comprise a logical sub-unit for communication, e.g., to facilitate data transfer from or to the device 2. However, a primary function of the control circuitry 23 is to control the aerosolization of aerosol generating material, as described in more detail below. It will be appreciated the functionality of the control circuitry 23 can be provided in various different ways, for example using one or more suitably programmed programmable computer(s) and/or one or more suitably configured application-specific integrated circuit(s)/circuitry/chip(s)/chipset(s) configured to provide the desired functionality. The control circuitry 23 is connected to the power supply 23 and receives power from the power source 22 and may be configured to distribute or control the power supply to other components of the aerosol provision device 2.

In the described implementation, the aerosol provision device 2 further comprises a receptacle 25 which is arranged to receive an aerosol generating article 4, such as the consumable described herein.

The receptacle 25 is suitable sized to removably receive the article 4 therein. Although not shown, the device 2 may comprise a hinged door or removable part of the outer housing 21 to permit access to the receptacle 25 such that a user may insert and/or remove the article 4 from the receptacle 25. The hinged door or removable part of the outer housing 21 may also act to retain the article 4 within the receptacle 25 when closed. When the aerosol generating article 4 is exhausted or the user simply wishes to switch to a different aerosol generating article 4, the aerosol generating article 4 may be removed from the aerosol provision device 2 and a replacement aerosol generating article 4 positioned in the receptacle 25 in its place. Alternatively, the device 2 may include a permanent opening that communicates with the receptacle 25 and through which the article 4 can be inserted into the receptacle 25. In such embodiments, a retaining mechanism for retaining the article 4 within the receptacle 25 of the device 2 may be provided.

As seen in FIG. 1, the device 2 comprises a number of aerosol generating components 24. In the described implementation, the aerosol generating components 24 are heating elements 24, and more specifically resistive heating elements 24. Resistive heating elements 24 receive an electrical current and convert the electrical energy into heat. The resistive heating elements 24 may be formed from, or comprise, any suitable resistive heating material, such as NiChrome (NiCr), which generates heat upon receiving an electrical current. In one implementation, the heating elements 24 may comprise an electrically insulating substrate on which resistive tracks are disposed. However, as discussed above and as should be appreciated, the heating elements may be any suitable form of heating element.

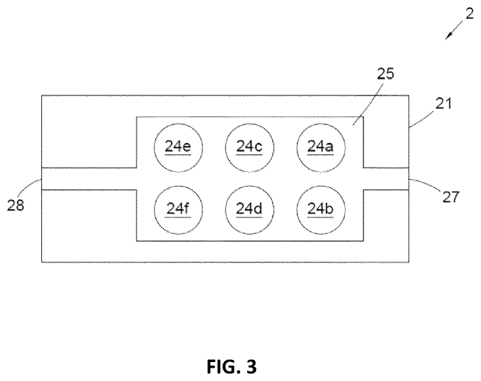

FIG. 3 is a cross-sectional, top-down view of the aerosol provision device 2 showing the arrangement of the heating elements 24 in more detail. In FIGS. 1 and 3, the heating elements 24 are positioned such that a surface of the heating element 24 forms a part of the surface of the receptacle 25. That is, an outer surface of the heating elements 24 is flush with the inner surface of the receptacle. More specifically, the outer surface of the heating element 24 that is flush with the inner surface of the receptacle 25 is a surface of the heating element 24 that is heated (i.e., its temperature increases) when an electrical current is passed through the heating element 24.

The heating elements 24 are arranged such that, when the article 4 is received in the receptacle 25, each heating element 24 aligns with a corresponding discrete portion of aerosol generating material 44. Hence, in this example, six heating elements 24 are arranged in two by three array broadly corresponding to the arrangement of the two by three array of the six discrete portions of aerosol generating material 44 shown in FIGS. 2A to 2C. However, as discussed above, the number of heating elements 24 may be different in different embodiments, for example there may be 8, 10, 12, 14, etc. heating elements 24. In some embodiments, the number of heating elements 24 is greater than or equal to six but no greater than 20.

More specifically, the heating elements 24 are labelled 24a to 24f in FIG. 3, and it should be appreciated that each heating element 24 is arranged to align with a corresponding portion of aerosol generating material 44 as denoted by the corresponding letter following the references 24/44. Accordingly, each of the heating elements 24 can be individually activated to heat a corresponding portion of aerosol generating material 44.

While the heating elements 24 are shown flush with the inner surface of the receptacle 25, in other embodiments the heating elements 24 may protrude into the receptacle 25. In either case, the article 4 contacts the surfaces of the heating elements 24 when present in the receptacle 25 such that heat generated by the heating elements 24 is conducted to the aerosol generating material 44 through the carrier component 42. In some embodiments, to improve the heat-transfer efficiency, the receptacle may comprise components which apply a force to the surface of the carrier component 42 so as to press the carrier component 42 onto the heater elements 24, thereby increasing the efficiency of heat transfer via conduction to the aerosol generating material 44. Additionally or alternatively, the heater elements 24 may be configured to move in the direction towards/away from the article 4, and may be pressed into the surface of carrier component 42 that does not comprise the aerosol generating material 44.

In use, the device 2 (and more specifically the control circuitry 23) is configured to deliver power to the heating elements 24 in response to a user input. Broadly speaking, the control circuitry 23 is configured to selectively apply power to the heating elements 24 to subsequently heat the corresponding portions of aerosol generating material 44 to generate aerosol. When a user inhales on the device 2 (i.e., inhales at mouthpiece end 26), air is drawn into the device 2 through air inlet 27, into the receptacle 25 where it mixes with the aerosol generated by heating the aerosol generating material 44, and then to the user's mouth via air outlet 28. That is, the aerosol is delivered to the user through mouthpiece end 26 and air outlet 28.

The device 2 of FIG. 1 includes a touch-sensitive panel 29 and an inhalation sensor 30. Collectively, the touch-sensitive panel 29 and inhalation sensor 30 act as mechanisms for a receiving a user input to cause the generation of aerosol, and thus may more broadly be referred to as user input mechanisms. The received user input may be said to be indicative of a user's desire to generate aerosol.

The touch-sensitive panel 29 may be a capacitive touch sensor and can be operated by a user of the device 2 placing their finger or another suitably conductive object (for example a stylus) on the touch-sensitive panel. In the described implementation, the touch-sensitive panel includes a region which can be pressed by a user to start aerosol generation. The control circuitry 23 may be configured to receive signaling from the touch-sensitive panel 29 and to use this signaling to determine if a user is pressing (i.e. activating) the region of the touch-sensitive panel 29. If the control circuitry 23 receives this signaling, then the control circuitry 23 is configured to supply power from the power source 22 to one or more of the heating elements 24. Power may be supplied for a predetermined time period (for example, three seconds) from the moment a touch is detected, or in response to the length of time the touch is detected for. In other embodiments, the touch sensitive panel 29 may be replaced by a user actuatable button or the like.

The inhalation sensor 30 may be a pressure sensor or microphone or the like configured to detect a drop in pressure or a flow of air caused by the user inhaling on the device 2. The inhalation sensor 30 is located in fluid communication with the air flow pathway (that is, in fluid communication with the air flow path between inlet 27 and outlet 28). In a similar manner as described above, the control circuitry 23 may be configured to receive signaling from the inhalation sensor and to use this signaling to determine if a user is inhaling on the aerosol provision system 1. If the control circuitry 23 receives this signaling, then the control circuitry 23 is configured to supply power from the power source 22 to one or more of the heating elements 24. Power may be supplied for a predetermined time period (for example, three seconds) from the moment inhalation is detected, or in response to the length of time the inhalation is detected for.

In the described example, both the touch-sensitive panel 29 and inhalation sensor 30 detect the user's desire to begin generating aerosol for inhalation. The control circuitry 23 may be configured to only supply power to the heating element 24 when signaling from both the touch-sensitive panel 29 and inhalation sensor 30 are detected. This may help prevent inadvertent activation of the heating elements 24 from accidental activation of one of the user input mechanisms. However, in other embodiments, the aerosol provision system 1 may have only one of a touch sensitive panel 29 and an inhalation sensor 30.

These aspects of the operation of the aerosol provision system 1 (i.e., puff detection and touch detection) may in themselves be performed in accordance with established techniques (for example using conventional inhalation sensor and inhalation sensor signal processing techniques and using conventional touch sensor and touch sensor signal processing techniques).

Turning now to the operation of the device 2, in response to detecting the signaling from either one or both of the touch-sensitive panel 29 and inhalation sensor 30, the control circuitry 23 is configured to supply power to one or more of the heating elements 24.

In some embodiments, the control circuitry 23 may be configured to generate an alert signal which signifies the end of use of the article 4, for example when each of the heating elements 24 has been activated a predetermined number of times, or when a given heating element 24 has been activated a predetermined number of times and/or for a given cumulative activation time and/or with a given cumulative activation power. In FIG. 1, the device 2 includes an end of use indicator 31 which in this implementation is an LED. However, in other embodiments, the end of use indicator 31 may comprise any mechanism which is capable of supplying an alert signal to a user; that is, the end of use indicator 31 may be an optical element to deliver an optical signal, a sound generator to deliver an aural signal, and/or a vibrator to deliver a haptic signal. In some embodiments, the indicator 31 may be combined or otherwise provided by the touch-sensitive panel (e.g., if the touch-sensitive panel includes a display element). The device 2 may prevent subsequent activation of the device 2 when the alert signal is being output. The alert signal may be switched off, and the control circuitry 23 reset, when the user replaces the article 4 and/or switches off the alert signal via a manual means such as a button (not shown).

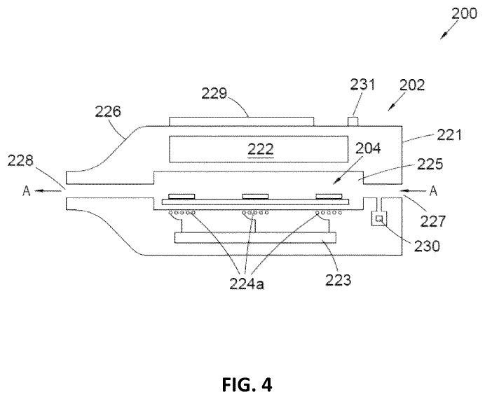

FIG. 4 is a cross-sectional view through a schematic representation of an aerosol provision system 200 in accordance with another embodiment of the disclosure. The aerosol provision system 200 includes components that are broadly similar to those described in relation to FIG. 1; however, the reference numbers have been increased by 200. For efficiency, the components having similar reference numbers should be understood to be broadly the same as their counterparts in FIG. 1 and FIGS. 2A to 2C unless otherwise stated.

The aerosol provision device 202 comprises an outer housing 221, a power source 222, control circuitry 223, induction work coils 224a, a receptacle 225, a mouthpiece end 226, an air inlet 227, an air outlet 228, a touch-sensitive panel 229, an inhalation sensor 230, and an end of use indicator 231.

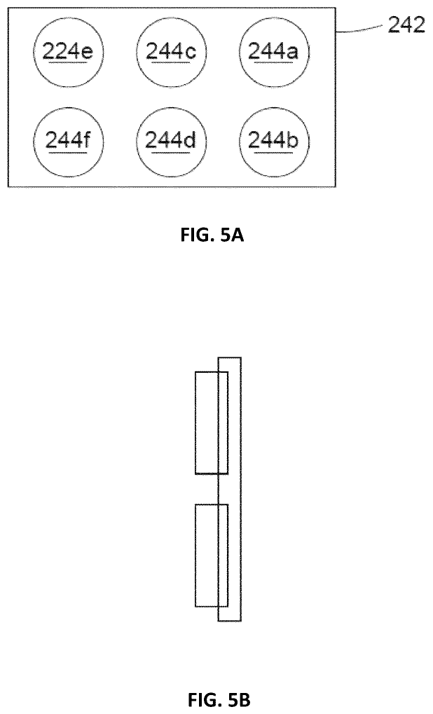

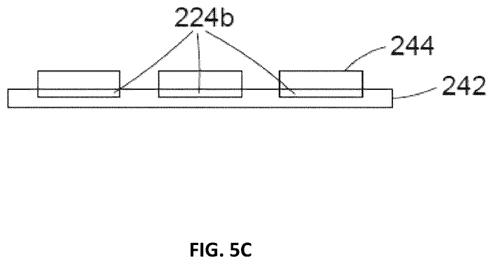

The aerosol generating article 204 comprises a carrier component 242, aerosol generating material 244, and susceptor elements 244b, as shown in more detail in FIGS. 5A to 5C. FIG. 5A is a top-down view of the article 4, FIG. 5B is an end-on view along the longitudinal (length) axis of the article 4, and FIG. 5C is a side-on view along the width axis of the article 4.

FIG. 4 and FIGS. 5A to 5C represent an aerosol provision system 200 which uses induction to heat the aerosol generating material 244 to generate an aerosol for inhalation. In the described embodiment, the aerosol generating component 224 is formed of two parts; namely, induction work coils 224a which are located in the aerosol provision device 202 and susceptors 224b which are located in the aerosol generating article 204. Accordingly, in this described embodiment, each aerosol generating component 224 comprises elements that are distributed between the aerosol generating article 204 and the aerosol provision device 202.

Induction heating is a process in which an electrically conductive object, referred to as a susceptor, is heated by penetrating the object with a varying magnetic field. The process is described by Faraday's law of induction and Ohm's law. An induction heater may comprise an electromagnet and a device for passing a varying electrical current, such as an alternating current, through the electromagnet. When the electromagnet and the object to be heated are suitably relatively positioned so that the resultant varying magnetic field produced by the electromagnet penetrates the object, one or more eddy currents are generated inside the object. The object has a resistance to the flow of electrical currents. Therefore, when such eddy currents are generated in the object, their flow against the electrical resistance of the object causes the object to be heated. This process is called Joule, ohmic, or resistive heating.

The heating material may be an electrically conductive material, so that penetration thereof with a varying magnetic field causes induction heating of the heating material. The heating material may be magnetic material, so that penetration thereof with a varying magnetic field causes magnetic hysteresis heating of the heating material. The heating material may be both electrically conductive and magnetic, so that the heating material is heatable by both heating mechanisms. Magnetic hysteresis heating is a process in which an object made of a magnetic material is heated by penetrating the object with a varying magnetic field. A magnetic material can be considered to comprise many atomic-scale magnets, or magnetic dipoles. When a magnetic field penetrates such material, the magnetic dipoles align with the magnetic field. Therefore, when a varying magnetic field, such as an alternating magnetic field, for example as produced by an electromagnet, penetrates the magnetic material, the orientation of the magnetic dipoles changes with the varying applied magnetic field. Such magnetic dipole reorientation causes heat to be generated in the magnetic material.

When an object is both electrically conductive and magnetic, penetrating the object with a varying magnetic field can cause both Joule heating and magnetic hysteresis heating in the object. Moreover, the use of magnetic material can strengthen the magnetic field, which can intensify the Joule heating.

In some embodiments, the heating material comprises a susceptor. A susceptor is material that is heatable by penetration with a varying magnetic field, such as an alternating magnetic field. In the described embodiment, the susceptors 224b are formed from an aluminium foil, although it should be appreciated that other metallic and/or electrically conductive materials may be used in other embodiments. As seen in FIG. 5C, the carrier component 242 comprises a number of susceptors 224b which correspond in size and location to the discrete portions of aerosol generating material 244 disposed on the surface of the carrier component 242. That is, the susceptors 224b have a similar width and length to the discrete portions of aerosol generating material 244. The susceptors are shown embedded in the carrier component 242. However, in other embodiments, the susceptors 224b may be placed on the surface of the carrier component 242.

The aerosol provision device 202 comprises a plurality of induction work coils 224a shown schematically in FIG. 4. The work coils 224a are shown adjacent the receptacle 225 and are generally flat coils arranged such that the rotational axis about which a given coil is wound extends into the receptacle 225 and is broadly perpendicular to the plane of the carrier component 242 of the article 204. The exact windings are not shown in FIG. 4 and it should be appreciated that any suitable induction coil may be used. The control circuitry 223 comprises a mechanism to generate an alternating current which is passed to any one or more of the induction coils 224a. The alternating current generates an alternating magnetic field, as described above, which in turn causes the corresponding susceptor(s) 224b to heat up. The heat generated by the susceptor(s) 224b is transferred to the portions of aerosol generating material 244 accordingly.

As described above in relation to FIG. 1 and FIGS. 2A to 2C, the control circuitry 223 is configured to supply current to the work coils 224a in response to receiving signaling from the touch sensitive panel 229 and/or the inhalation sensor 230. Any of the techniques for selecting which heating elements 24 are heated by control circuitry 23 as described previously may analogously be applied to selecting which work coils 224a are energized (and thus which portions of aerosol generating material 244 are subsequently heated) in response to receiving signaling from the touch sensitive panel 229 and/or the inhalation sensor 230 by control circuitry 223 to generate an aerosol for user inhalation.

Although the above has described an induction heating aerosol provision system where the work coils 224a and susceptors 224b are distributed between the article 204 and device 202, an induction heating aerosol provision system may be provided where the work coils 224a and susceptors 224b are located solely within the device 202. For example, with reference to FIG. 4, the susceptors 224b may be provided above the induction work coils 224a and arranged such that the susceptors 224b contact the lower surface of the carrier component 242 (in an analogous way to the aerosol provision system 1 shown in FIG. 1).

Thus, FIG. 4 describes a more concrete implementation where induction heating may be used in an aerosol provision device 202 to generate aerosol for user inhalation to which the techniques described in the present disclosure may be applied.

Although the above has described a system in which an array of aerosol generating components 24 (e.g., heater elements) are provided to energize the discrete portions of aerosol generating material, in other embodiments, the article 4 and/or an aerosol generating component 24 may be configured to move relative to one another. That is, there may be fewer aerosol generating components 24 than discrete portions of aerosol generating material 44 provided on the carrier component 42 of the article 4, such that relative movement of the article 4 and aerosol generating components 24 is required in order to be able to individually energize each of the discrete portions of aerosol generating material 44. For example, a movable heating element 24 may be provided within the receptacle 25 such that the heating element 24 may move relative to the receptacle 25. In this way, the movable heating element 24 can be translated (e.g., in the width and length directions of the carrier component 42) such that the heating element 24 can be aligned with respective ones of the discrete portions of aerosol generating material 44. This approach may reduce the number of aerosol generating components 42 required while still offering a similar user experience.

Although the above has described embodiments where the device 2 can be configured or operated using the touch-sensitive panel 29 mounted on the device 2, the device 2 may instead be configured or controlled remotely. For example, the control circuitry 23 may be provided with a corresponding communication circuitry (e.g., Bluetooth) which enables the control circuitry 23 to communicate with a remote device such as a smartphone. Accordingly, the touch-sensitive panel 29 may, in effect, be implemented using an App or the like running on the smartphone. The smartphone may then transmit user inputs or configurations to the control circuitry 23 and the control circuitry 23 may be configured to operate on the basis of the received inputs or configurations.

Although the above has described embodiments in which an aerosol is generated by energizing (e.g., heating) aerosol generating material 44 which is subsequently inhaled by a user, it should be appreciated in some embodiments that the generated aerosol may be passed through or over an aerosol modifying component to modify one or more properties of the aerosol before being inhaled by a user. For example, the aerosol provision device 2, 202 may comprise an air permeable insert (not shown) which is inserted in the airflow path downstream of the aerosol generating material 44, 244 (for example, the insert may be positioned in the outlet 28, 228). The insert may include a material which alters any one or more of the flavor, temperature, particle size, nicotine concentration, etc. of the aerosol as it passes through the insert before entering the user's mouth. For example, the insert may include tobacco or treated tobacco. Such systems may be referred to as hybrid systems. The insert may include any suitable aerosol modifying material, which may encompass the aerosol generating materials described above. Although the above has described embodiments in which the aerosol provision device 2, 202 comprises an end of use indicator 31, 231, it should be appreciated that the end of use indicator 31, 231 may be provided by another device remote from the aerosol provision device 2, 202. For example, in some embodiments, the control circuitry 23, 223 of the aerosol provision device 2, 202 may comprise a communication mechanism which allows data transfer between the aerosol provision device 2, 202 and a remote device such as a smartphone or smartwatch, for example. In these embodiments, when the control circuitry 23, 223 determines that the article 4, 204 has reached its end of use, the control circuitry 23, 223 is configured to transmit a signal to the remote device, and the remote device is configured to generate the alert signal (e.g., using the display of a smartphone). Other remote devices and other mechanisms for generating the alert signal may be used as described above.

In some embodiments, the article 4, 204 may comprise an identifier, such as a readable bar code or an RFID tag or the like, and the aerosol provision device 2, 202 comprises a corresponding reader. When the article is inserted into the receptacle 25, 225 of the device 2, 202, the device 2, 202 may be configured to read the identifier on the article 4, 204. The control circuitry 23, 223 may be configured to either recognize the presence of the article 4, 204 (and thus permit heating and/or reset an end-of-life indicator) or identify the type and/or the location of the portions of the aerosol generating material relative to the article 4, 204. This may affect which portions the control circuitry 23, 223 aerosolizes and/or the way in which the portions are aerosolized, e.g., via adjusting the aerosol generation temperature and/or heating duration. Any suitable technique for recognizing the article 4, 204 may be employed.

In addition, when the portions of aerosol generating material are provided on a carrier component 42, 242, the portions may, in some embodiments, include weakened regions, e.g., through holes, vents or areas of relatively thinner aerosol generating material, in a direction approximately perpendicular to the plane of the carrier component 42, 242. This may be the case when the hottest part of the aerosol generating material is the area directly contacting the carrier component (in other words, in scenarios where the heat is applied primarily to the surface of the aerosol generating material that contacts the carrier component 42, 242). Accordingly, the through holes may provide channels for the generated aerosol to escape and be released to the environment rather than causing a potential build-up of aerosol between the carrier component 42, 242 and the aerosol generating material 44, 244. Such build-up of aerosol can reduce the heating efficiency of the system as the build-up of aerosol can, in some embodiments, cause a lifting of the aerosol generating material from the carrier component 42, 242 thus decreasing the efficiency of the heat transfer to the aerosol generating material. Each portion of aerosol generating material may be provided with one of more weakened regions as appropriate.

Thus, there has been described a method of generating aerosol from a consumable or an aerosol generating article which comprises discrete portions of aerosol generating material as described herein. The method comprises heating a first portion of aerosol generating material comprising to generate aerosol from the first aerosol generating material and heating a second aerosol generating material to generate aerosol from the second aerosol generating material. Heating of the first aerosol generating material and heating the second aerosol generating material may be arranged to occur at substantially the same time or may occur consecutively. Also described is an aerosol provision device and an aerosol provision system.

While the above-described embodiments have in some respects focussed on some specific example aerosol provision systems, it will be appreciated the same principles can be applied for aerosol provision systems using other technologies. That is to say, the specific manner in which various aspects of the aerosol provision system function are not directly relevant to the principles underlying the examples described herein.

### Method of Providing an Aerosol

In another aspect is provided a method of providing an aerosol containing an active agent as disclosed herein, the method comprising heating a discrete portion of aerosol generating material, the aerosol generating material comprising an aerosol former, a binder and an active agent (e.g., a substituted 3-(1-methylpyrrolidin-2-yl)pyridine), each as disclosed herein.

The heating may comprise heating the discrete portion of aerosol generating material to a temperature of less than or equal to 350° C., such as from about 220° C. to about 280° C. or from about 250° C. to about 280° C.

In each case, the aerosol provided may comprise a specific dose or amount of the active agent (e.g., a substituted 3-(1-methylpyrrolidin-2-yl)pyridine, 3-(azetidin-2-yl)pyridine, or 3-(azetidin-2-ylmethoxy)pyridine). In some embodiments, about 95% or more, such as about 99% or more or about 100% (i.e. all or substantially all) of the active agent will be aerosolized, for example when the aerosol generating material is heated to a temperature of less than or equal to 350° C., such as from about 220° C. to about 280° C. or from about 250° C. to about 280° C., for at least 1 second, such as from about 1 second to about 5 seconds or from about 1 second to about 3 seconds. In one embodiment, the above-mentioned dose or amount of active agent will be aerosolized when the aerosol generating material is heated to a temperature of 280° C. for a period of 3 seconds.

In some embodiments, the aerosol is generated using a non-combustible aerosol provision system as described herein. In some embodiments, the method comprises heating the aerosol generating material to a temperature of less than or equal to 350° C. In some embodiments, the method comprises heating the aerosol generating material to a temperature of from about 220° C. to about 280° C. In some embodiments, the method comprises heating at least a portion of the aerosol generating material to a temperature of from about 220° C. to about 280° C. over a session of use.

A “session of use” as used herein refers to a single period of use of the non-combustible aerosol provision system by a user. The session of use begins at the point at which power is first supplied to at least one heating unit present in the heating assembly. The device will be ready for use after a period of time has elapsed from the start of the session of use. The session of use ends at the point at which no power is supplied to any of the heating elements. The end of the session of use may coincide with the point at which the article is depleted (the point at which the total particulate matter yield (mg) in each puff would be deemed unacceptably low by a user). The session will have a duration of a plurality of puffs. Said session may have a duration less than 7 minutes, or 6 minutes, or 5 minutes, or 4 minutes and 30 seconds, or 4 minutes, or 3 minutes and 30 seconds. In some embodiments, the session of use may have a duration of from 2 to 5 minutes, or from 3 to 4.5 minutes, or 3.5 to 4.5 minutes, or suitably 4 minutes. A session may be initiated by the user actuating a button or switch on the device, causing at least one heating element to begin rising in temperature.

The amount or dose of the active agent in the aerosol will therefore be almost or substantially the same as the amount of the active agent in the aerosol generating material. The amount of active agent inhaled by a user can therefore be accurately controlled.

Many modifications and other embodiments of the disclosure will come to mind to one skilled in the art to which this disclosure pertains having the benefit of the teachings presented in the foregoing descriptions and the associated figures. Therefore, it is to be understood that the disclosure is not to be limited to the specific embodiments disclosed herein and that modifications and other embodiments are intended to be included within the scope of the appended claims. Although specific terms are employed herein, they are used in a generic and descriptive sense only and not for purposes of limitation.

1. A consumable comprising one or more discrete portions of aerosol generating material, the aerosol generating material comprising an aerosol former, a binder, and an active agent, the active agent comprising at least a compound having a structure according to Formula I, Formula II, or Formula III: 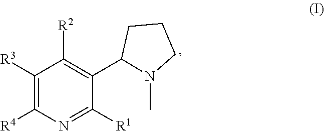
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or 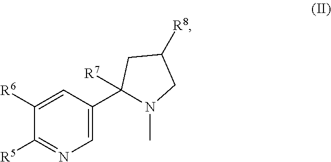
wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C1-C3 alkyl; and
at least one of R⁷ and R⁸ is not hydrogen; or 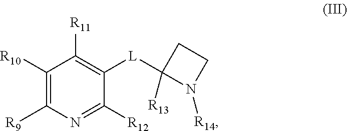
wherein
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

2. The consumable of claim 1, wherein the active agent comprises a compound having a structure according to Formula I, wherein R¹, R², and R³ are each H.

3. The consumable of claim 2, wherein R⁴ is optionally substituted C₁-C₆ alkyl, F, Cl, Br, OCH₃, OEt, or CN.

4. The consumable of claim 3, wherein R⁴ is C₁-C₃ alkyl, such as wherein R⁴ is CH₃.

5. The consumable of claim 1, wherein the active agent comprises a compound having a structure according to Formula II, wherein R⁵ and R⁶ are H; R⁷ is CH₃; and R⁸ is H.

6. The consumable of claim 1, wherein the active agent comprises a compound having a structure according to Formula II, wherein R⁵, R⁶ and R⁷ are H, and R⁸ is CH₃.

7. The consumable of claim 1, wherein each discrete portion comprises from about 0.01 to about 5 mg of the active agent.

8. The consumable of claim 1, wherein the aerosol generating material comprises from about 1 to about 5 wt % of the active agent.

9. The consumable of claim 1, wherein the discrete portions of aerosol generating material are located on a carrier.

10. The consumable of claim 9, wherein the carrier is formed from metal foil, paper, carbon paper, grease-proof paper, ceramic, a carbon allotrope, plastic, cardboard, wood, or a combination thereof.

11. The consumable of claim 1, comprising at least two discrete portions of aerosol generating material, and wherein a total amount of the active agent in any given discrete portion is within 10% of a total amount of active agent in any of the other discrete portions.

12. The consumable of claim 1, wherein the aerosol generating material comprises from about 1 to about 80% by weight of the aerosol former.

13. The consumable of claim 1, wherein the aerosol former comprises glycerol, propylene glycol, 1,3-propanediol, diethylene glycol, triethylene glycol, tetraethylene glycol, 1,3-butylene glycol, erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, or a combination thereof.

14. The consumable of claim 1, wherein the aerosol generating material comprises from about 0.5% to about 60% of the binder by weight.

15. The consumable of claim 1, wherein the binder comprises an alginate, a pectin, a starch, a cellulose derivative, a gum, silica, clay, polyvinyl alcohol, or a combination thereof.

16. The consumable of claim 1, wherein the aerosol generating material further comprises a filler in an amount by weight up to about 20%.

17. The consumable of claim 1, wherein the filler is a fibrous organic material, a cellulosic material, or a combination thereof.

18. A non-combustible aerosol provision system comprising a non-combustible aerosol provision device and the consumable of claim 1.

19. The non-combustible aerosol provision system of claim 18, wherein the non-combustible aerosol provision device is a heat-not-burn device.
